SEQUENCE LISTING (6 sequences)

SEQ ID NO: 1 - RNA, 7862 nt, synthetic construct
  misc_feature 1..7862: note = WEEV-based srRNA vector derived from strain Imperial, derived from GenBank accession no. GQ287641, without coding sequence of a gene of interest
  source 1..7862: mol_type = other RNA
gatagggcacggtatagaggaacctaccctacagactaatcgatccaatatggaaagaattcacgttgacttagatgctgacagcccgtatgtcaagtcgttacagcggagctttccacaatttgagatcgaagcaaggcaggtcactgacaatgaccatgccaatgccagagcgttttcgcatgtggcaacaaagctcattgagagcgaagtcgaccgggaccaagttatcttggacattggaagtgcgcccgtcagacatgcacattccaatcaccgctatcattgtatctgccctatgataagcgctgaagacccggacagactacaacggtatgcagaaagacttaagaaaagtgacattaccgacaagaacatagcctctaaggcggcagacctgctggaagtcatgtcaacaccagacgcagagactccatctctgtgtatgcacacagacgccacgtgtaggtactttggaagtgtagcagtataccaagatgtgtacgcagtccatgcaccgacatcaatctaccaccaggcgcttaaaggagttaggacaatttactggataggctttgacacgaccccttttatgtacaaaaacatggcaggttcttaccctacttacaacacgaactgggctgacgagagagtattggaagcacgtaacattggcctcggtaactcagatcttcaggagagcaggcttggaaacctctcaatccttaggaagaagaggctccaacctactaataagatcatattctcggttggttcaacaatctacacagaagatagatcactgttacgtagctggcatcttccaaacgtgttccacttgaaaggaaagtctaacttcacaggtagatgtgggaccattgtcagctgtgaagggtacgtcataaaaaaaattacaatcagcccaggactatacggtaaagttgagaacttggcgtccacaatgcatcgcgagggtttcttgagttgcaaagtcacagatacgttgcgcggcgagagggtttcttttgctgtgtgtacgtatgtaccagccacactttgcgatcagatgacagggattctggcaactgacgttagtgtggatgacgcacaaaaactattggttgggctcaaccaaaggattgtcgtcaatggtaggacgcaaagaaatactaacacaatgcagaactatctattaccagtggtcgcccaggcgttttccaggtgggcgcgtgaacatcgtgccgacttggacgacgagaaagaactaggggtgcgggagcgcactcttactatgggctgctgctgggctttcaagacccagaaaatcacatccatctacaagaagcctggtacgcaaacaattaagaaagtacctgccgtctttgactcatttgtgattccacgccttaccagccacgggctcgatatgggcttccgccgtaggctcaagctgctgcttgaaccaactgtcaaacccgcaccggctattacaatggccgatgtggagcatctgcgtggcttacagcaagaagctgaagaagtggctgcagcggaagagatcagagaagccctgccgcccttgctccctgaaatagaaaaagagaccgtagaggcagaagtagacctcattatgcaagaggcaggagcaggtagcgtggagacaccacgaggacacatcagggtgacaagttacccaggcgaagagaagattgggtcttacgctgtactttcaccccaggcggtattgaatagtgaaaaactggcgtgtatccacccattggcggaacaagtactggtaatgactcacaaaggtagggctgggagatacaaggtcgagccataccacggtaaggtcattgtaccagaagggacggcggtccctgttcaagacttccaggcattgagtgagagcgctacgatcgttttcaacgagagggagttcgtaaacagatacctgcatcacatcgcaatcaacggaggagcgctaaacactgacgaagagtactataagactgtaaagactcaggacacagactcagaatacgtcttcgacgttgacgcacgaaagtgtgttaagcgagaagacgcaggtccgttgtgcctaaccggtgatctggtagatccgccatttcacgagtttgcgtacgagagtcttaggacgcgaccagcagcacctcacaaagtcccaaccattggagtctatggagtgccaggttcgggtaaatctggaatcatcaaaagcgctgtgaccaagaaagatctggttgtgagtgcgaagaaggaaaactgcgcagaaatcatcagggatgtaaggaggatgagatgtatggatgttgctgctaggactgtagattctgtgctgctgaatggggttaagcaccccgttaacactctgtacattgatgaggcatttgcctgccatgcagggacgctgctggcactgattgccatcgtcaaacctaagaaagtggtattgtgcggggacccaaaacaatgcggcttctttaacatgatgtgcctgaaagtacattttaaccatgacatatgcactgaagtgtaccataaaagcatctctaggaggtgcacacagactgtaaccgctatcgtctccacgctcttttacgacaagcgaatgaagacggttaacccatgtgctgacaaaatcatcatagataccacagggaccacaaagccgcacaaagatgatctgattctaacctgtttcagaggatgggtgaaacagctacagattgactataaaaatcatgaaatcatgactgcggctgcatcgcaaggacttacgcggaaaggcgtttatgctgtcaggtacaaagtcaacgagaatccactctactcgcagacttctgagcacgtgaacgtgttacttacacgcacagaaaaacgcattgtctggaagacgctagctggtgatccctggataaagatacttacagctaaatattccggggatttcacggcttcattggacgactggcagcgagaacatgatgccattatggcacgcgttcttgataagccgcagacagctgatgtgttccagaataaggtgaacgtctgctgggcgaaggctctagagccagtcttggccacggccaacattgtgctgacgagacagcagtgggagacgttgcacccattcaagcatgacagagcgtactcacctgaaatggcactgaacttcttttgcaccaggttctttggagtagacctggacagtgggttgttttccgctcctaccgtcgcacttacttatagggatcagcactgggataactcgccagggaagaacatgtatgggcttaatagagaggtagcaaaggagttgtcacggcgatatccgtgcatcacaaaagcggttgacacaggcagggtagctgatataaggaataataccatcaaggactactctccaacaattaatgtggttccattaaatcgtcggttaccccactcgttgatcgttgaccacaaaggacagggtacaactgatcacagcggattcctatctaagatgaagggcaaatctgtgttggtgatcggcgatcctatcagcattccagggaagaaagtggagtccatgggtccattgcccactaataccatcaggtgtgatctagatttgggaatacctagccatgtcggtaaatatgacattatatttgtcaatgttaggaccccgtataagaaccatcactaccaacagtgcgaggatcacgctatccaccacagcatgttaacgtgtaaggctgtccaccacctgaacactggcggaacatgtgtggctatagggtatgggcttgctgatcgcgcaaccgagaatatcatcactgcggtagcgcgctcatttaggtttacccgtgtctgtcagcctaagaacactgccgaaaatactgaggttctcttcgtgttcttcggcaaggacaacggcaaccacacacatgaccaggacagactcgctgtagtgcttgacaacatctaccaagggtcaaccaggtacgaggcagggagagctccagcgtacagagtgatcagaggtgacattagcaagagcgctgaccaagctatcgttaatgctgctaatagcaaaggtcaaccaggttccggagtgtgcggtgcactgtaccgaaaatggccggctgcttttgatagacagccaatagctgtcgggacggctagacttgtgaagcacgaaccgctcatcatacatgctgtaggacccaatttttctaagatgccggaaccggagggcgaccttaagctcgcagctgcctacatgagcatagcgtcaatcgtcaacgctgaacggattactaaaatatcagtaccgctactgtcaaccggcatctattctggtggcaaagatcgagtgatgcaatcattgcatcacctgttcactgctttcgacactacggatgccgatgtcaccatatattgcttggataaacaatgggagaccaggataatcgaggccattcaccgcaaagaaagcgtcgaaatcctggatgatgacaagccggtagacattgacttggtcagggtccatccaaacagcgctttggcaggcagacctggttactccgtcaatgagggcaagctgtattcatacctggaaggtacacgattccatcagaccgccaaggacattgccgaaatccatgcaatgtggcccaacaaatctgaggctaatgagcagatttgcttgtacatcctgggtgagagtatgtccagcatccgctccaaatgcccagtagaggagtcagaggcgtctgctccacctcacacactgccgtgcctgtgtaattacgctatgacggctgagcgcgtatacaggttgcgctctgcgaagaaagaacagttcgccgtatgctcatcattcctgttgccgaagtacaggatcacaggcgtgcagaagctacaatgcagcaaaccagtcctgttttcaggcgtcgtaccgccggctgtacaccccaggaagtacgcggaaataattctagaaacgccaccaccgccagtaacgacaaccgtaatatgtgaacccactgtgccagaacgtatacccagtccggcgatttctagagcaccaagtgcggaatcactgctatcttttagcggcgtctcgttctctagctctgccacacgctcgtcaaccgcctggagcgactatgacaggcggtttgtggttacagctgacgtgcatcaagcgaacacatctacgtggagcatccctagtgctcctggcttggacgtccaaataccttctgacgtcagtgattcccactggagtgttccgagtgcatcaggcttcgaagtgagaacaccatctgtacaggacctaactgcggagtgtgcaaagcctcgtgggctggccgaaataatgcaagacttcaatacagcccctttccagtttctttcggaccacagaccagtaccggcaccacggagacgccccatcccatcacctagatcgacggtttccgcacctccagttccaaagccacgcaggactaagtaccaacaaccaccaggagtcgctagagcgatctcagaagcggagttggacgagtacatccgtcaacactccaattgacggtatgaagcgggagcgtatattttctcatcggaaacaggccaaggtcaccttcaacagaaatcagtacgtcaatgtaaactacaagaacctatattggaccgggccgtccatgagaagtattacgccccgcgcctcgatctcgaaagagagaaaatgttacagaagaaattgcaattatgtgcctctgaaggaaatagaagcaggtatcaatcacgaaaagtagaaaatatgaaagcaattacagcggagcgactcatttctggattgggcacatacctatcatcagaagtgaatcctgtcgagtgttacagagtcaactatcctgtaccaatctactcgtcaacggtaattaacaggtttacatctgcagaggtcgcggttaaaacgtgcaacttagttatccaagagaattaccctacagtagccagttattgtataacagatgaatacgatgcgtatcttgacatggtggacggcgcatcgtgctgtctagatacagccactttttgtccggctaaactgagaagctacccaaagaagcatagctatttgcagccagagataagatcagccgtcccatcgcctatacagaatacattacaaaatgtattggctgcagctactaaaaggaattgcaacgttacccaaatgcgagaattacctgtcttagattcggcggcatttaacgttgattgtttcaagaaatacgcatgcaatgatgagtactgggatacctttcgcgataaccctattcggctaactacagagaacgttacgcaatatgtgacaaagctgaaagggccgaaagcagcagcattgtttgcgaaaactcacaatttaaaaccgttgcaggagataccaatggatcaattcgtcatggatctaaaaagagatgtcaaagttactcccggcacgaaacatacagaggagcggcctaaggtgcaggttattcaggctgcagacccccttgctaccgcttacctttgcgggatccaccgggaattagtccgtagactgaacgctgtgcttctgccgaatatccatactctcttcgacatgtcagcggaagattttgatgcgattattgctggacatttccaccacggcgacccagtattggaaacggacatcgcgtcgtttgataaaagcgaagacgacgctatcgccatttcggcgatgatgatccttgaggacttaggcgtcgaccaaccgctcttagatttgatagaggcggcgttcggcaatatcacatctgtgcacctacctacaggaacgaggtttaaatttggtgccatgatgaaatccggcatgttcttaacgctgtttgtcaacacactggtcaatatcatgattgctagcagagtactacgtgaacggttaaccacgtcagcgtgcgcggcctttatcggcgacgataacatagtgcatggtgtcgtctccgacaccttgatggcggagagatgcgccacttggctgaacatggaagtaaaaattattgatgcagtcattggtatcaaagcaccctacttctgcgggggatttatcctggtggaccagataacaggcacagcctgcagggtcgcagaccctctaaaaaggctttttaagcttggaaaaccgttgtcagttgaggacacccaagactgcgaccgccgccgggcactgcatgatgaagcaatgcgatggaacagaatcggaattacggacgagttggtgaaggccgtagaatccagatacgagatcatactggcaggcctgatcatcacgtctctgtccacgttagccgaaagcgttaagaacttcaagagcataagagggagcccaatcaccctctacggctgacctaaataggtgacgtagtagacacgcacctacccatcgccatactggagacgtggaggagaaccctggacctactagtgaccgctacgccccaatgacccgaccagctgagcgcggacactgacatagcggtaaaaactcgatgtacttccgaggaagcgtggtgcataatgccacgcgccgcttgacactaaaactcgatgtatttccgaggaagcacagtgcataatgctgtgcagtgtcacattaatcgcatatcacactatatattaacaacactatatcacttttataagactcactatgggtctctaatatacactacacatattttacttaaaaacactatacacactttataagttcttttataatttttcttttgtttttattttgtttttaaaatttc

SEQ ID NO: 2 - RNA, 9562 nt, synthetic construct
  misc_feature 1..9562: note = WEEV-based srRNA vector derived from strain Imperial, derived from GenBank accession no. GQ287641, including coding sequence of HA protein from influenza
  source 1..9562: mol_type = other RNA
gatagggcacggtatagaggaacctaccctacagactaatcgatccaatatggaaagaattcacgttgacttagatgctgacagcccgtatgtcaagtcgttacagcggagctttccacaatttgagatcgaagcaaggcaggtcactgacaatgaccatgccaatgccagagcgttttcgcatgtggcaacaaagctcattgagagcgaagtcgaccgggaccaagttatcttggacattggaagtgcgcccgtcagacatgcacattccaatcaccgctatcattgtatctgccctatgataagcgctgaagacccggacagactacaacggtatgcagaaagacttaagaaaagtgacattaccgacaagaacatagcctctaaggcggcagacctgctggaagtcatgtcaacaccagacgcagagactccatctctgtgtatgcacacagacgccacgtgtaggtactttggaagtgtagcagtataccaagatgtgtacgcagtccatgcaccgacatcaatctaccaccaggcgcttaaaggagttaggacaatttactggataggctttgacacgaccccttttatgtacaaaaacatggcaggttcttaccctacttacaacacgaactgggctgacgagagagtattggaagcacgtaacattggcctcggtaactcagatcttcaggagagcaggcttggaaacctctcaatccttaggaagaagaggctccaacctactaataagatcatattctcggttggttcaacaatctacacagaagatagatcactgttacgtagctggcatcttccaaacgtgttccacttgaaaggaaagtctaacttcacaggtagatgtgggaccattgtcagctgtgaagggtacgtcataaaaaaaattacaatcagcccaggactatacggtaaagttgagaacttggcgtccacaatgcatcgcgagggtttcttgagttgcaaagtcacagatacgttgcgcggcgagagggtttcttttgctgtgtgtacgtatgtaccagccacactttgcgatcagatgacagggattctggcaactgacgttagtgtggatgacgcacaaaaactattggttgggctcaaccaaaggattgtcgtcaatggtaggacgcaaagaaatactaacacaatgcagaactatctattaccagtggtcgcccaggcgttttccaggtgggcgcgtgaacatcgtgccgacttggacgacgagaaagaactaggggtgcgggagcgcactcttactatgggctgctgctgggctttcaagacccagaaaatcacatccatctacaagaagcctggtacgcaaacaattaagaaagtacctgccgtctttgactcatttgtgattccacgccttaccagccacgggctcgatatgggcttccgccgtaggctcaagctgctgcttgaaccaactgtcaaacccgcaccggctattacaatggccgatgtggagcatctgcgtggcttacagcaagaagctgaagaagtggctgcagcggaagagatcagagaagccctgccgcccttgctccctgaaatagaaaaagagaccgtagaggcagaagtagacctcattatgcaagaggcaggagcaggtagcgtggagacaccacgaggacacatcagggtgacaagttacccaggcgaagagaagattgggtcttacgctgtactttcaccccaggcggtattgaatagtgaaaaactggcgtgtatccacccattggcggaacaagtactggtaatgactcacaaaggtagggctgggagatacaaggtcgagccataccacggtaaggtcattgtaccagaagggacggcggtccctgttcaagacttccaggcattgagtgagagcgctacgatcgttttcaacgagagggagttcgtaaacagatacctgcatcacatcgcaatcaacggaggagcgctaaacactgacgaagagtactataagactgtaaagactcaggacacagactcagaatacgtcttcgacgttgacgcacgaaagtgtgttaagcgagaagacgcaggtccgttgtgcctaaccggtgatctggtagatccgccatttcacgagtttgcgtacgagagtcttaggacgcgaccagcagcacctcacaaagtcccaaccattggagtctatggagtgccaggttcgggtaaatctggaatcatcaaaagcgctgtgaccaagaaagatctggttgtgagtgcgaagaaggaaaactgcgcagaaatcatcagggatgtaaggaggatgagatgtatggatgttgctgctaggactgtagattctgtgctgctgaatggggttaagcaccccgttaacactctgtacattgatgaggcatttgcctgccatgcagggacgctgctggcactgattgccatcgtcaaacctaagaaagtggtattgtgcggggacccaaaacaatgcggcttctttaacatgatgtgcctgaaagtacattttaaccatgacatatgcactgaagtgtaccataaaagcatctctaggaggtgcacacagactgtaaccgctatcgtctccacgctcttttacgacaagcgaatgaagacggttaacccatgtgctgacaaaatcatcatagataccacagggaccacaaagccgcacaaagatgatctgattctaacctgtttcagaggatgggtgaaacagctacagattgactataaaaatcatgaaatcatgactgcggctgcatcgcaaggacttacgcggaaaggcgtttatgctgtcaggtacaaagtcaacgagaatccactctactcgcagacttctgagcacgtgaacgtgttacttacacgcacagaaaaacgcattgtctggaagacgctagctggtgatccctggataaagatacttacagctaaatattccggggatttcacggcttcattggacgactggcagcgagaacatgatgccattatggcacgcgttcttgataagccgcagacagctgatgtgttccagaataaggtgaacgtctgctgggcgaaggctctagagccagtcttggccacggccaacattgtgctgacgagacagcagtgggagacgttgcacccattcaagcatgacagagcgtactcacctgaaatggcactgaacttcttttgcaccaggttctttggagtagacctggacagtgggttgttttccgctcctaccgtcgcacttacttatagggatcagcactgggataactcgccagggaagaacatgtatgggcttaatagagaggtagcaaaggagttgtcacggcgatatccgtgcatcacaaaagcggttgacacaggcagggtagctgatataaggaataataccatcaaggactactctccaacaattaatgtggttccattaaatcgtcggttaccccactcgttgatcgttgaccacaaaggacagggtacaactgatcacagcggattcctatctaagatgaagggcaaatctgtgttggtgatcggcgatcctatcagcattccagggaagaaagtggagtccatgggtccattgcccactaataccatcaggtgtgatctagatttgggaatacctagccatgtcggtaaatatgacattatatttgtcaatgttaggaccccgtataagaaccatcactaccaacagtgcgaggatcacgctatccaccacagcatgttaacgtgtaaggctgtccaccacctgaacactggcggaacatgtgtggctatagggtatgggcttgctgatcgcgcaaccgagaatatcatcactgcggtagcgcgctcatttaggtttacccgtgtctgtcagcctaagaacactgccgaaaatactgaggttctcttcgtgttcttcggcaaggacaacggcaaccacacacatgaccaggacagactcgctgtagtgcttgacaacatctaccaagggtcaaccaggtacgaggcagggagagctccagcgtacagagtgatcagaggtgacattagcaagagcgctgaccaagctatcgttaatgctgctaatagcaaaggtcaaccaggttccggagtgtgcggtgcactgtaccgaaaatggccggctgcttttgatagacagccaatagctgtcgggacggctagacttgtgaagcacgaaccgctcatcatacatgctgtaggacccaatttttctaagatgccggaaccggagggcgaccttaagctcgcagctgcctacatgagcatagcgtcaatcgtcaacgctgaacggattactaaaatatcagtaccgctactgtcaaccggcatctattctggtggcaaagatcgagtgatgcaatcattgcatcacctgttcactgctttcgacactacggatgccgatgtcaccatatattgcttggataaacaatgggagaccaggataatcgaggccattcaccgcaaagaaagcgtcgaaatcctggatgatgacaagccggtagacattgacttggtcagggtccatccaaacagcgctttggcaggcagacctggttactccgtcaatgagggcaagctgtattcatacctggaaggtacacgattccatcagaccgccaaggacattgccgaaatccatgcaatgtggcccaacaaatctgaggctaatgagcagatttgcttgtacatcctgggtgagagtatgtccagcatccgctccaaatgcccagtagaggagtcagaggcgtctgctccacctcacacactgccgtgcctgtgtaattacgctatgacggctgagcgcgtatacaggttgcgctctgcgaagaaagaacagttcgccgtatgctcatcattcctgttgccgaagtacaggatcacaggcgtgcagaagctacaatgcagcaaaccagtcctgttttcaggcgtcgtaccgccggctgtacaccccaggaagtacgcggaaataattctagaaacgccaccaccgccagtaacgacaaccgtaatatgtgaacccactgtgccagaacgtatacccagtccggcgatttctagagcaccaagtgcggaatcactgctatcttttagcggcgtctcgttctctagctctgccacacgctcgtcaaccgcctggagcgactatgacaggcggtttgtggttacagctgacgtgcatcaagcgaacacatctacgtggagcatccctagtgctcctggcttggacgtccaaataccttctgacgtcagtgattcccactggagtgttccgagtgcatcaggcttcgaagtgagaacaccatctgtacaggacctaactgcggagtgtgcaaagcctcgtgggctggccgaaataatgcaagacttcaatacagcccctttccagtttctttcggaccacagaccagtaccggcaccacggagacgccccatcccatcacctagatcgacggtttccgcacctccagttccaaagccacgcaggactaagtaccaacaaccaccaggagtcgctagagcgatctcagaagcggagttggacgagtacatccgtcaacactccaattgacggtatgaagcgggagcgtatattttctcatcggaaacaggccaaggtcaccttcaacagaaatcagtacgtcaatgtaaactacaagaacctatattggaccgggccgtccatgagaagtattacgccccgcgcctcgatctcgaaagagagaaaatgttacagaagaaattgcaattatgtgcctctgaaggaaatagaagcaggtatcaatcacgaaaagtagaaaatatgaaagcaattacagcggagcgactcatttctggattgggcacatacctatcatcagaagtgaatcctgtcgagtgttacagagtcaactatcctgtaccaatctactcgtcaacggtaattaacaggtttacatctgcagaggtcgcggttaaaacgtgcaacttagttatccaagagaattaccctacagtagccagttattgtataacagatgaatacgatgcgtatcttgacatggtggacggcgcatcgtgctgtctagatacagccactttttgtccggctaaactgagaagctacccaaagaagcatagctatttgcagccagagataagatcagccgtcccatcgcctatacagaatacattacaaaatgtattggctgcagctactaaaaggaattgcaacgttacccaaatgcgagaattacctgtcttagattcggcggcatttaacgttgattgtttcaagaaatacgcatgcaatgatgagtactgggatacctttcgcgataaccctattcggctaactacagagaacgttacgcaatatgtgacaaagctgaaagggccgaaagcagcagcattgtttgcgaaaactcacaatttaaaaccgttgcaggagataccaatggatcaattcgtcatggatctaaaaagagatgtcaaagttactcccggcacgaaacatacagaggagcggcctaaggtgcaggttattcaggctgcagacccccttgctaccgcttacctttgcgggatccaccgggaattagtccgtagactgaacgctgtgcttctgccgaatatccatactctcttcgacatgtcagcggaagattttgatgcgattattgctggacatttccaccacggcgacccagtattggaaacggacatcgcgtcgtttgataaaagcgaagacgacgctatcgccatttcggcgatgatgatccttgaggacttaggcgtcgaccaaccgctcttagatttgatagaggcggcgttcggcaatatcacatctgtgcacctacctacaggaacgaggtttaaatttggtgccatgatgaaatccggcatgttcttaacgctgtttgtcaacacactggtcaatatcatgattgctagcagagtactacgtgaacggttaaccacgtcagcgtgcgcggcctttatcggcgacgataacatagtgcatggtgtcgtctccgacaccttgatggcggagagatgcgccacttggctgaacatggaagtaaaaattattgatgcagtcattggtatcaaagcaccctacttctgcgggggatttatcctggtggaccagataacaggcacagcctgcagggtcgcagaccctctaaaaaggctttttaagcttggaaaaccgttgtcagttgaggacacccaagactgcgaccgccgccgggcactgcatgatgaagcaatgcgatggaacagaatcggaattacggacgagttggtgaaggccgtagaatccagatacgagatcatactggcaggcctgatcatcacgtctctgtccacgttagccgaaagcgttaagaacttcaagagcataagagggagcccaatcaccctctacggctgacctaaataggtgacgtagtagacacgcacctacccatcgccatactggagacgtggaggagaaccctggacctaatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagctgagcgcggacactgacatagcggtaaaaactcgatgtacttccgaggaagcgtggtgcataatgccacgcgccgcttgacactaaaactcgatgtatttccgaggaagcacagtgcataatgctgtgcagtgtcacattaatcgcatatcacactatatattaacaacactatatcacttttataagactcactatgggtctctaatatacactacacatattttacttaaaaacactatacacactttataagttcttttataatttttcttttgtttttattttgtttttaaaatttc

SEQ ID NO: 3 - DNA, 29 bp, synthetic construct
  misc_feature 1..29: note = 5' adaptor sequence
  source 1..29: mol_type = other DNA
ctggagacgtggaggagaaccctggacct

SEQ ID NO: 4 - DNA, 29 bp, synthetic construct
  misc_feature 1..29: note = 3' adaptor sequence
  source 1..29: mol_type = other DNA
gaccgctacgccccaatgacccgaccagc

SEQ ID NO: 5 - DNA, 18 bp, synthetic construct
  misc_feature 1..18: note = bacteriophage T7 RNA polymerase promoter
  source 1..18: mol_type = other DNA
taatacgactcactatag

SEQ ID NO: 6 - DNA, 29 bp, synthetic construct
  misc_feature 1..29: note = T7 terminator sequence
  source 1..29: mol_type = other DNA
aacccctctctaaacggaggggttttttt

(19) United States
(12) Patent Application Publication
(10) Pub. No.: US 2025/0257329 A1
(43) Pub. Date: Aug. 14, 2025

(54) MODIFIED WESTERN EQUINE ENCEPHALITIS VIRUSES AND USES THEREOF

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US)

(21) Appl. No.: 18/858,163

(22) PCT Filed: Apr. 18, 2023
(86) PCT No.: PCT/US2023/065898

§ 371 Date: Oct. 18, 2024

Related U.S. Application Data
(60) Provisional application No. 63/332,606, filed on Apr. 19, 2022.

(51) Int. Cl. C12N 7/00; A01K 67/0275; C12N 5/071; C12N 15/86; C12P 21/00

(52) U.S. Cl. CPC C12N 7/00; A01K 67/0275; C12N 5/0686; C12N 15/86; C12P 21/00; C12N 2770/36121; C12N 2770/36143; C12N 2830/50

(57) ABSTRACT

The present disclosure relates to the field of molecular virology, including nucleic acid molecules comprising modified viral genomes or self-replicating RNAs, pharmaceutical compositions containing the same, and the use of such nucleic acid molecules and compositions for production of desired products in cell cultures or in a living body. Also provided are methods for eliciting a pharmacodynamic effect in a subject in need thereof, as well as methods for preventing and/or treating various health conditions.

### CROSS-REFERENCE TO RELATED APPLICATIONS

This application claims the benefit of priority to U.S. Provisional Patent Application Ser. No. 63/332,606, filed on Apr. 19, 2022. The disclosure of the above-referenced application is herein expressly incorporated by reference it its entirety, including any drawings.

### INCORPORATION OF THE SEQUENCE LISTING

The material in the accompanying Sequence Listing is hereby incorporated by reference into this application. The accompanying Sequence Listing XML file, named 2023 Apr. 13 Sequence_Listing_ST26 058462-511001WO.xml, was created on Apr. 13, 2023, and is 26,362 bytes in size.

### FIELD

The present disclosure relates to the field of molecular virology and immunology, and particularly relates to nucleic acid molecules encoding modified viral genomes and replicons, e.g., self-replicating RNA (srRNA) molecules, pharmaceutical compositions containing the same, and the use of such nucleic acid molecules and compositions for production of desired products in cell cultures or in a living body. Also provided are methods for eliciting a pharmacodynamic effect in a subject in need thereof, as well as methods for preventing and/or treating various health conditions.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, gene therapy vectors, and drug delivery vehicles.

For example, many viral-based expression vectors have been deployed for expression of heterologous proteins in cultured recombinant cells. For example, the application of modified viral vectors for gene expression in host cells continues to expand. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

However, it has been reported that host cells can develop intricate and powerful mechanisms to detect and counter pathogen invasion. It has been further reported that viruses, particularly pathogenic viruses, have evolved with host cells to combat these cellular defenses to infection and replication. As a result of infection, many host cells shut down cellular protein translation machinery in order to control viral replication and/or viral production of progeny that can potentially spread to additional cells. This phenomenon is generally termed as the “innate immune response.” Infected cells also send out danger signals to other cells, locally and systemically, to set up an antiviral state and control the infection. Although these cellular antiviral systems benefit the host cells, they can also negatively impact self-replicating RNA (srRNA) designed to express beneficial vaccine antigens or therapeutic agents. For example, if a cell detects an srRNA expressing a beneficial protein and activates its innate immune defense mechanisms, the expression of the beneficial protein in such cell can be impacted and the efficacy of the srRNA can be compromised.

Therefore, there is still a need for more efficient methods and systems for expressing products of interest in srRNA-based expression platforms.

### SUMMARY

The present disclosure relates generally to the development of immuno-therapeutics, such as recombinant nucleic acids constructs and pharmaceutical compositions including the same for use in the prevention and management of various health conditions such as proliferative disorders and microbial infection. In particular, as described in greater detail below, some embodiments of the disclosure provide nucleic acid constructs containing sequences that encode a modified genome or replicon, e.g., self-replicating RNA (srRNA) of the alphavirus Western Equine Encephalitis virus (WEEV) that is devoid at least a portion of the viral nucleic acid sequence encoding one or more structural proteins of the virus. Also disclosed are recombinant cells and transgenic animals that have been engineered to include one or more of the nucleic acid constructs disclosed herein, methods for producing a molecule of interest (e.g., a polypeptide of interest), pharmaceutical compositions including one or more of the following: (a) a nucleic acid construct of the disclosure, (b) a polypeptide of the disclosure, (c) a recombinant cell of the disclosure. Further provided in particular aspects of the disclosure are compositions and methods for eliciting a pharmacodynamic effect in a subject in need thereof, and/or for the prevention and/or treatment of various health conditions, including proliferative disorders (e.g., cancers) and infections.

In one aspect of the disclosure, provided herein are nucleic acid constructs including a nucleic acid sequence encoding a modified Western Equine Encephalitis virus (WEEV) genome or replicon, e.g., self-replicating RNA (srRNA), wherein the modified WEEV genome or replicon, e.g., srRNA is devoid of at least a portion of the nucleic acid sequence encoding one or more viral structural proteins.

Non-limiting exemplary embodiments of the nucleic acid constructs of the disclosure can include one or more of the following features. In some embodiments, the modified viral genome or replicon, e.g., srRNA is devoid of a substantial portion of the nucleic acid sequence encoding one or more viral structural proteins. In some embodiments, the modified viral genome or replicon, e.g., srRNA includes no nucleic acid sequence encoding viral structural proteins. In some embodiments, the nucleic acid molecules of the disclosure further include one or more expression cassettes, wherein each of the expression cassettes includes a promoter operably linked to a heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter.

In some embodiments, at least one nonstructural protein (nsP), or a portion thereof, of the modified WEEV genome or replicon, e.g., srRNA is heterologous relative to the remainder of the modified WEEV genome or replicon, e.g., srRNA. In some embodiments, the modified WEEV genome or replicon, e.g., srRNA further includes a nucleic acid sequence encoding a heterologous nsP or a portion thereof.

In some embodiments, the nucleic acid constructs of the disclosure further include one or more untranslated regions (UTRs). In some embodiments, at least one of the UTRs is a heterologous UTR.

In some embodiments, at least one of the expression cassettes includes a coding sequence for a gene of interest (GOI). In some embodiments, the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the coding sequence of the GOI is optimized for expression at a level higher than the expression level of a reference coding sequence. In some embodiments, the coding sequence of the GOI is optimized for enhanced RNA stability.

In some embodiments, the nucleic acid constructs of the disclosure include a nucleic acid sequence having at least 80%, at least 85%, at least 90%, at least 95%, at least 96%, at least 97%, at least 98%, at least 99%, or 100% sequence identity to the nucleic acid sequence of SEQ ID NO: 1 or SEQ ID NO: 2.

In one aspect, provided herein are recombinant cells including a nucleic acid construct as disclosed herein. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is an insect cell. In some embodiments, the recombinant cell is a mosquito cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), a canine kidney cell (MDCK), a buffalo rat liver cell (BRL 3A), a human lung cell (W138), a human liver cell (Hep G2), a mouse mammary tumor (MMT 060562), a TRI cell, a FS4 cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell. Also provided, in a related aspect, are cell cultures that include at least one recombinant cell as disclosed herein and a culture medium.

In another aspect, provided herein are transgenic animals including a nucleic acid construct as described herein. In some embodiments, the transgenic animal is a vertebrate animal or an invertebrate animal. In some embodiments, the transgenic animal is a mammalian. In some embodiments, the transgenic mammalian is a non-human mammalian. In some embodiments, the transgenic animal is an insect. In some embodiments, the transgenic insect is a transgenic mosquito. In another aspect, provided herein are methods for producing a polypeptide of interest, wherein the methods include (i) rearing a transgenic animal as disclosed herein; or (ii) culturing a recombinant cell including a nucleic acid construct as disclosed herein under conditions wherein the transgenic animal or recombinant cell produces the polypeptide encoded by the GOI.

In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is an insect. In some embodiments, the insect is a mosquito. In some embodiments, the subject is a mammalian subject. In some embodiments, the mammalian subject is a human subject. In yet another aspect, provided herein are recombinant polypeptides produced by a method of the disclosure.

In yet another aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient. In some embodiments, provided herein are compositions that formulated in a liposome, a lipid-based nanoparticle (LNP), a polymer nanoparticle, a polyplex, a viral replicon particle (VRP), a microsphere, an immune stimulating complex (ISCOM), a conjugate of bioactive ligand, or a combination of any thereof. In some embodiments, the compositions are immunogenic compositions. In some embodiments, the immunogenic compositions are formulated as a vaccine. In some embodiments, the immunogenic compositions are substantially non-immunogenic to a subject. In some embodiments, the pharmaceutical compositions are formulated as an adjuvant. In some embodiments, the pharmaceutical compositions are formulated for one or more of intranasal administration, intrathecal administration, transdermal administration, intraperitoneal administration, intramuscular administration, intratracheal administration, intranodal administration, intratumoral administration, intraarticular administration, intravenous administration, subcutaneous administration, intravaginal administration, intraocular administration, rectal administration, and oral administration.

In another aspect, provided herein are methods for eliciting a pharmacodynamic effect in a subject in need thereof, the method includes administering to the subject a composition including: a) a nucleic acid construct of the disclosure; b) a recombinant cell of the disclosure; c) a recombinant polypeptide of the disclosure; and/or d) a pharmaceutical composition of the disclosure. In some embodiments, the pharmacodynamic effect includes one or more of the following: immunogenicity effect, a biomarker response, a therapeutic effect, a prophylactic effect, a desired effect, an undesired effect, an adverse effect, and effect in a disease model. In some embodiments, the pharmacodynamic effect includes eliciting an immune response in the subject.

In yet another aspect, provided herein are methods for preventing and/or treating a health condition in a subject in need thereof, the method includes prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure. In some embodiments, the administered composition elicits pharmacodynamic effect. In some embodiments, the pharmacodynamic effect includes eliciting an immune response in the subject.

Non-limiting exemplary embodiments of the methods of the disclosure can include one or more of the following features. In some embodiments, the condition is a proliferative disorder or a microbial infection. In some embodiments, the subject has or is suspected of having a condition associated with proliferative disorder or a microbial infection. In some embodiments, the administered composition results in an increased production of interferon in the subject. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In yet another aspect, provided herein are kits for eliciting a pharmacodynamic response, eliciting an immune response, and/or for the prevention and/or treatment of a health condition or a microbial infection, the kit including: a) a nucleic acid construct of the disclosure; b) a recombinant cell of the disclosure; c) a recombinant polypeptide of the disclosure; and/or d) a pharmaceutical composition of the disclosure.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is graphical representation of a non-limiting example of a modified WEEV genome design in accordance with some embodiments of the disclosure, in which the nucleic acid sequence encoding viral structural proteins of the original virus have been completely deleted. The modified WEEV design described in this figure contains native 5′ UTR and 3′ UTR derived from the WEEV strain Imperial, and further contains a heterologous gene of interest (GOI) placed under control of a 26S subgenomic promoter. Coding sequences for the non-structural proteins nsP1, nsP2, nsP3, and nsP4 are shown.

FIGS. 2A-2B are graphical illustrations of non-limiting exemplary WEEV srRNA designs in accordance with some embodiments of the disclosure, in which the sequence encoding the modified WEEV genome from Imperial strain was incorporated into plasmid DNA vectors (FIG. 2A), which also included coding sequences for an exemplary gene of interest (GOI), e.g., hemagglutinin precursor (HA) of the influenza A virus H5N1 (FIG. 2B).

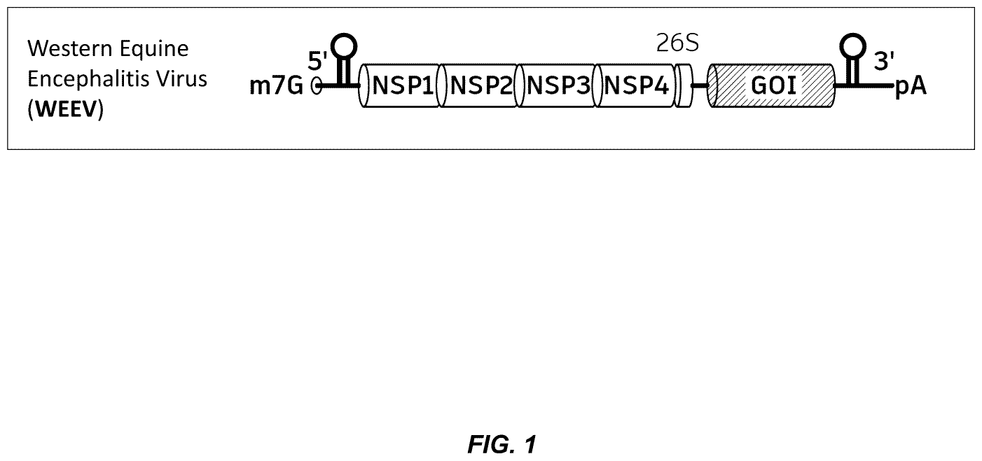

### DETAILED DESCRIPTION OF THE DISCLOSURE

Provided herein are, inter alia, viral expression systems with superior expression potential which are suitable for expressing heterologous molecules such as, for example, vaccines and therapeutic polypeptides, in recombinant cells. For example, some embodiments of the disclosure relate to nucleic acid constructs such as, e.g. expression constructs and vectors, containing a modified genome or replicon, e.g., srRNA of a Western Equine Encephalitis virus (WEEV) in which at least some of its original viral sequence encoding structural proteins has been deleted. Also provided in some embodiments of the disclosure are viral-based expression vectors including one or more expression cassettes encoding heterologous polypeptide. Further provided are recombinant cells that are genetically engineered to include one or more of the nucleic acid molecules disclosed herein. Biomaterials and recombinant products derived from such recombinant cells are also within the scope of the application. Also provided are compositions and methods useful for eliciting a pharmacodynamic effect in a subject in need thereof, as well as methods for preventing and/or treating various health conditions.

Self-replicating RNAs (srRNAs) based on RNA viruses (e.g., alphaviruses) can be used as robust expression systems. For example, it has been reported that an advantage of using alphaviruses such as WEEV as viral expression vectors is that they can direct the synthesis of large amounts of heterologous proteins in recombinant host cells. Among other advantages, polypeptides such as therapeutic single chain antibodies may be most effective if expressed at high levels in vivo. In addition, for producing recombinant antibodies purified from cells in culture (ex vivo), high protein expression from a srRNA may increase overall yields of the antibody product. Furthermore, if the protein being expressed is a vaccine antigen, high level expression may induce the most robust a pharmacodynamic effect in vivo.

Alphaviruses utilize motifs contained in their UTRs, structural regions, and non-structural regions to impact their replication in host cells. These regions also contain mechanism to evade host cell innate immunity. However, significant differences among alphavirus species have been reported. For example, New World and Old World Alphaviruses have evolved different components to exploit stress granules, JAK-STAT signaling, FXR, and G3BP proteins within cells for assembly of viral replication complexes. Which part of the genome contains these components also varies between Alphaviruses. For example, bypassing activation of PKR and subsequent phosphorylation of EIF2alpha is done via the downstream loop in some Old World Alphaviruses such as Sindbis, but bypassing this pathway is thought to be done via nsP4 in Chikungunya, which lacks a recognizable DLP. In addition, beyond variation between individual Alphaviruses, there are often differences within strains of Alphaviruses as well that can account for changes in characteristics such as virulence. As an example, sequence variations between North American and South American strains of the New World Eastern Equine Encephalitis virus (EEEV) alter the ability to modulate the STAT1 pathway leading to differential induction of Type I interferons and resulting changes in virulence. As a further example, mouse studies with a set of North American WEEV isolates with less than 2.7% genomic sequence divergence had outcomes ranging from zero to complete lethality.

Given the differential presence of host cell attenuating factors in non-structural and structural regions of Alphaviruses, deleting the structural genes to allow for heterologous gene expression in synthetic vectors will have varied impacts on individual vectors. Synthetic replicons with different host attenuating factors in the non-structural regions will differentially excel at the induction of immune responses to heterologous genes that are expressed. For example, while shutoff of host cell functions has been linked to nsP2 in Old World viruses, in New World viruses (e.g., EEEV, VEEV, and WEEV) this activity has been primarily associated with the capsid protein (C), which is deleted in part or in full in synthetic vectors. The hypervariable domain (HVD) of nsP3 proteins have host-interactions that are specific for each alphavirus. Among others, EEEV nsP3 has been shown to interact with cellular FXR and G3BP protein families, DDX3, S100A4, IKKβ, PGAM5, and cytoskeletal reorganization and vesicle trafficking proteins. There are few studies that detail WEEV non-structural proteins specifically, however genomic sequencing of WEEV reveals that it is a result of recombination between ancestors of EEEV and SINV. The amino acid identity of WEEV nsPs was reported to have 80%+ identity to EEEV nsPs, however replacing EEEV nsPs with WEEV nsPs results in attenuated chimeras, demonstrating that the changes in nucleotide and coding sequence retain some essential activities to the virus life cycle, but bear significant differences in biological activity. The known and undescribed mechanisms that EEEV and WEEV nsPs contribute to their wide range of pathogenicities suggest that WEEV-based srRNA vectors could make distinct, advantaged vectors for expression of heterologous proteins for vaccine or biotherapeutic applications. The advantages that these previously undescribed srRNA vectors confer has been completely unexplored and unpredicted.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a composition of the disclosure, e.g., nucleic acid constructs (for example, replicon constructs, e.g., srRNA constructs), recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “construct” refers to a recombinant molecule, e.g., recombinant nucleic acid or polypeptide, including one or more isolated nucleic acid sequences or amino acid sequences from heterologous sources. For example, polypeptide constructs can be chimeric polypeptide molecules in which two or more amino acid sequences of different origin are operably linked to one another in a single polypeptide construct. Similarly, nucleic acid constructs can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single-stranded or double-stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Nucleic acid constructs of the present disclosure can include the necessary elements to direct expression of a nucleic acid sequence of interest that is also contained in the construct. Such elements may include control elements such as a promoter that is operably linked to (so as to direct transcription of) the nucleic acid sequence of interest, and optionally includes a polyadenylation sequence.

In some embodiments of the disclosure, one or more nucleic acid constructs may be incorporated (e.g., inserted) within a single nucleic acid molecule, such as a single vector, or can be incorporated (e.g., inserted) within two or more separate nucleic acid molecules, such as two or more separate vectors. The term “vector” is used herein to refer to a nucleic acid molecule or sequence capable of transferring or transporting another nucleic acid molecule. Thus, the term “vector” encompasses both DNA-based vectors and RNA-base vectors. The term “vector” includes cloning vectors and expression vectors, as well as viral vectors and integrating vectors. An “expression vector” is a vector that includes a regulatory region, thereby capable of expressing DNA sequences and fragments in vitro, ex vivo, and/or in vivo. In some embodiments, a vector may include sequences that direct autonomous replication in a cell such as, for example a plasmid (DNA-based vector) or a self-replicating RNA vector. In some embodiments, a vector may include sequences sufficient to allow integration into host cell DNA. Useful vectors include, for example, plasmids (e.g., DNA plasmids or RNA plasmids), transposons, cosmids, bacterial artificial chromosomes, and viral vectors. In some embodiments, the vector of the disclosure can be single-stranded vector (e.g., ssDNA or ssRNA). In some embodiments, the vector of the disclosure can be double-stranded vector (e.g., dsDNA or dsRNA). In some embodiments, a vector is a gene delivery vector. In some embodiments, a vector is used as a gene delivery vehicle to transfer a gene into a cell. In some embodiments, the vector of the disclosure is a self-replicating RNA (srRNA) vector.

In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a cell. Two or more constructs can be incorporated within a single nucleic acid molecule, such as a single vector, or can be incorporated within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or may be applied to, the complement of a query sequence. This definition includes sequence comparison performed by a BLAST algorithm wherein the parameters of the algorithm are selected to give the largest match between the respective sequences over the entire length of the respective reference sequences. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al., Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition of interest at the time of diagnosis or later. The term “non-human animals” includes all vertebrates, e.g., mammals, e.g., rodents, e.g., mice, livestock, domesticated animals and pets, non-human primates, and other mammals, such as e.g., sheep, cats, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximate. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

### Western Equine Encephalitis Virus (WEEV)

Western Equine Encephalitis virus (WEEV) is a mosquito-borne virus belonging to the genus Alphavirus which include a group of genetically, structurally, and serologically related viruses of the Togaviridae family and classified as a group IV positive-sense single-stranded RNA viruses. Currently, the alphavirus genus includes among others the Sindbis virus (SINV), the Semliki Forest virus (SFV), the Ross River virus (RRV), Venezuelan equine encephalitis virus (VEEV), and Eastern Equine Encephalitis virus (EEEV), which are all closely related and are able to infect various vertebrates such as mammalians, rodents, fish, avian species, and larger mammals such as humans and horses as well as invertebrates such as insects. One unusual feature of WEEV is that it is the descendant of an ancient recombination event between Sindbis virus (SINV)-like and eastern equine encephalitis virus (EEEV)-like ancestors.

The Alphavirus genus, including WEEV and EEEV, has been widely studied and the life cycle, mode of replication, etc., of these viruses are well characterized. More information in this regard can be found in, e.g., Corrin T. et al., Vector-Borne and Zoonotic Diseases, Vol. 21, No. 5, 2021. In addition, alphaviruses have been shown to replicate very efficiently in animal cells which makes them valuable as vectors for production of protein and nucleic acids in such cells. Transmission between species and individuals occurs mainly via mosquitoes making the alphaviruses a contributor to the collection of Arboviruses—or Arthropod-Borne Viruses.

Each of these alphaviruses has a single stranded RNA genome of positive polarity enclosed in a nucleocapsid surrounded by an envelope containing viral spike proteins. Alphavirus particles are enveloped, tend to be spherical (although slightly pleomorphic), and have an isometric nucleocapsid. Alphavirus genome is single-stranded RNA of positive polarity of approximately 11-12 kb in length, comprising a 5′ cap, a 3′ poly-A tail, and two open reading frames with a first frame encoding the nonstructural proteins with enzymatic function and a second frame encoding the viral structural proteins (e.g., the capsid protein CP, E1 glycoprotein, E2 glycoprotein, E3 protein and 6K protein). For instance, WEEV possesses a single-stranded, positive-sense RNA genome of approximately 11.5 kb including two open reading frames (ORFs) flanked by 5′- and 3′-untranslated regions (UTRs) and that is capped at the 5′ end and polyadenylated at the 3′ end. WEEV is transmitted primarily in agricultural habitats by its primary mosquito vector, Culex (Culex) tarsalis. Mammals can participate in a secondary cycle. Both humans and horses are thought to be dead-end hosts, although some equids, such as burros and ponies, develop low to moderate levels of viremia (slightly under 104 PFU/ml), which could allow these hosts to contribute to epizootic amplification. Human infections with WEEV range from no-noticeable symptoms (the vast majority) to a flu-like syndrome to life-threatening encephalitis and meningitis. Symptomatic infection typically includes a sudden onset with fever, headache, nausea, vomiting, anorexia, and malaise, followed by cognitive symptoms, weakness, and meningeal involvement.

The 5′ two-thirds of the alphavirus genome encodes a number of nonstructural proteins necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1-4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsP4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphavirus glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses, including WEEV, lose the peripheral protein E3, while in Semliki viruses it remains associated with the viral surface.

Similar to most alphaviruses, WEEV replication takes place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP proteins and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Self-Replicating RNA

As will be appreciated by the skilled artisan, the term “self-replicating RNA” refers to RNA molecule that contains all of the genetic information required for directing its own amplification or self-replication within a permissive cell. Therefore, srRNA is sometimes also referred to as “self-amplifying RNA” (saRNA). In some embodiments, the srRNA is a “replicon,” which can be a linear or circular section of DNA or RNA which replicates sequentially as a unit. Non-limiting examples of replicons include “replicon RNA” or “RNA replicon.” To direct its own replication, the srRNA generally (1) encodes polymerase, replicase, or other proteins which may interact with viral or host cell-derived proteins, nucleic acids or ribonucleoproteins to catalyze the RNA amplification process; and (2) contain cis-acting RNA sequences required for replication and transcription of the subgenomic RNA. These sequences may be bound during the process of replication to its self-encoded proteins, or non-self-encoded cell-derived proteins, nucleic acids or ribonucleoproteins, or complexes between any of these components. In some embodiments of the disclosure, the replicon, e.g., srRNA, is derived from an alphavirus, e.g., WEEV. In some embodiments of the disclosure, an alphavirus srRNA construct (e.g., srRNA, saRNA, or replicon molecule) generally contains the following elements: 5′ viral or defective-interfering RNA sequence(s) required in cis for replication, sequences coding for biologically active alphavirus non-structural proteins (e.g., nsP1, nsP2, nsP3, and nsP4), a subgenomic promoter (sg) for the subgenomic RNA (sgRNA), 3′ viral sequences required in cis for replication, and optionally a polyadenylate tract (poly(A)). In some instances, a subgenomic promoter (sg) that directs expression of a heterologous sequence can be included in the srRNA construct of the disclosure.

Further, the term srRNA molecule (e.g., srRNA, saRNA, or replicon molecule) generally refers to a molecule of positive polarity, or “message” sense, and the srRNA may be of length different from that of any known, naturally-occurring alphavirus. In some embodiments of the present disclosure, the srRNA does not contain at least a portion of the coding sequence for one or more of the alphavirus structural proteins; and/or sequences encoding structural genes can be substituted with heterologous sequences. In those instances, where the srRNA is to be packaged into a recombinant alphavirus particle, it can contain one or more sequences, so-called packaging signals, which serve to initiate interactions with alphavirus structural proteins that lead to particle formation.

Nucleic acid molecules of the present disclosure can be nucleic acid molecules of any length, including nucleic acid molecules that are generally between about 2 kb and 50 kb in length, for example between about 5 kb and about 40 kb, between about 5 kb and about 30 kb, between about 5 kb and about 20 kb, or between about 10 kb and about 50 kb, for example between about 15 kb to 30 kb, between about 20 kb and about 50 kb, between about 20 kb and about 40 kb, between about 5 kb and about 25 kb, or between about 30 kb and about 50 kb. In some embodiments, the nucleic acid molecules are at least 6 kb in length. In some embodiments, the nucleic acid molecules are between about 6 kb and about 20 kb. The replicon constructs (e.g., srRNA constructs) of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

### COMPOSITIONS OF THE DISCLOSURE

As described in greater detail below, one aspect of the present disclosure relates to nucleic acid constructs a nucleic acid sequence encoding a modified viral genome or srRNA, wherein the modified genome or srRNA is devoid of (e.g. does not include) at least a portion of the nucleic acid sequence encoding one or more structural proteins of the corresponding unmodified viral genome or srRNA. Some embodiments of the disclosure provide a modified alphavirus genome or srRNA in which the coding sequence for non-structural proteins nsP1, nsP2, nsP3, and nsP4 is present, however at least a portion of or the entire sequence encoding one or more structural proteins is absent. Also provided are recombinant cells and cell cultures that have been engineered to include a nucleic acid construct as disclosed herein.

### A. Nucleic Acid Constructs

As described in greater detail below, one aspect of the present disclosure relates to novel nucleic acid constructs including a nucleic acid sequence encoding a modified genome or srRNA of an alphavirus, such as Western Equine Encephalitis virus (WEEV). For example, in some embodiments, a modified alphavirus genome can include deletion(s), substitution(s), and/or insertion(s) in one or more of the genomic regions of the parent alphavirus genome.

Non-limiting exemplary embodiments of the nucleic acid constructs (for example, replicon constructs, e.g., srRNA constructs) of the disclosure can include one or more of the following features. In some embodiments, the nucleic acid constructs include a nucleic acid sequence encoding a modified WEEV genome or srRNA, wherein the modified WEEV genome or srRNA is devoid of at least a portion of the nucleic acid sequence encoding one or more structural proteins of the unmodified WEEV genome or srRNA, e.g., the modified WEEV genome or srRNA does not include at least a portion of the coding sequence for one or more of the WEEV structural proteins CP, E1, E2, E3, and 6K. Virulent and avirulent WEEV strains are both suitable. Non-limiting examples of WEEV strains suitable for the compositions and methods of the disclosure include WEEV California, McMillan, IMP181, Imperial, Imperial181, IMPR441, 71V-1658, AG80-646, BFS932, COA592, EP-6, E1416, BFS1703, BFS2005, BSF3060, BSF09997, CHLV53, KERN5547, 85452NM, Montana-64, S8-122, and TBT-235. Additional examples of WEEV strains suitable for the compositions and methods of the disclosure include 5614, 93A27, 93A30, 93A38, 93A79, B628 (Cl 15), CBA87, CNTR34, CO921356, Fleming, Lake43, PV012357A, PV02808A, PV72102, R02PV001807A, R02PV002957B, R02PV003422B, R05PV003422B, R0PV003814A and R0PV00384A. Additional suitable WEEV strains include, but are not limited to those described in Bergren N A et al., J. Virol. 88(16): 9260-9267 August 2014, and in the Virus Pathogen Resource website (ViPR; which is publicly available at www.viprbrc.org/brc/vipr_genome_search.spg?method=SubmitForm&blockId=868&decorator=toga). In some embodiments, the modified WEEV genome or srRNA is derived from WEEV strain Imperial.

Non-limiting exemplary embodiments of the nucleic acid constructs (for example, replicon constructs, e.g., srRNA constructs) of the disclosure can include one or more of the following features. In some embodiments, the modified viral genome or srRNA is devoid of at least a portion of the nucleic acid sequence encoding one or more of the viral structural proteins CP, E1, E2, E3, and 6K of the unmodified viral genome or srRNA. In some embodiments, the modified viral genome or srRNA is devoid of a portion of or the entire sequence encoding CP. In some embodiments, the modified viral genome or srRNA is devoid of a portion of or the entire sequence encoding E1. In some embodiments, the modified viral genome or srRNA is devoid of a portion of or the entire sequence encoding E2. In some embodiments, the modified viral genome or srRNA is devoid of a portion of or the entire sequence encoding E3. In some embodiments, the modified viral genome or srRNA is devoid of a portion of or the entire sequence encoding 6K. In some embodiments, the modified viral genome or srRNA is devoid of a portion of or the entire sequence encoding a combination of CP, E1, E2, E3, and 6K. Some embodiments of the disclosure provide a modified WEEV genome or srRNA in which the coding sequence for non-structural proteins nsP1, nsP2, nsP3, and nsP4 of the unmodified WEEV genome or srRNA is present, however at least a portion of or the entire sequence encoding one or more structural proteins (e.g., CP, E1, E2, E3, and 6K) of the WEEV genome or srRNA is absent. Some embodiments of the disclosure provide a modified WEEV genome or srRNA in which the coding sequence for non-structural proteins nsP1, nsP2, nsP3, and nsP4 of the unmodified WEEV genome or srRNA is present, however at least a portion of or the entire sequence encoding one or more structural proteins (e.g., CP, E1, E2, E3, and 6K) of the WEEV genome or srRNA is absent.

In some embodiments, the modified viral genome or srRNA is devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full-length nucleic acid sequence. As described above, the present disclosure provides nucleic acid molecules and constructs which are devoid of partial or complete nucleic acid sequences encoding one or more viral structural proteins. The skilled artisan, having the benefit of the sequences as disclosed herein, can readily use all or a substantial portion of the disclosed sequences for the compositions and methods of the disclosure. Accordingly, the present application comprises the complete sequences as disclosed herein, e.g., those set forth in the accompanying Sequence Listing, as well as substantial portions of those sequences as defined above.

In some embodiments, the modified viral genome or srRNA is devoid of the entire sequence encoding viral structural proteins, e.g., the modified viral genome or srRNA includes no nucleic acid sequence encoding the structural proteins of the viral unmodified genome or srRNA.

In some embodiments, the nucleic acid constructs of the disclosure further include one or more expression cassettes. In principle, the nucleic acid constructs disclosed herein can generally include any number of expression cassettes. In some embodiments, the nucleic acid constructs disclosed herein can include at least two, at least three, at least four, at least five, or at least six expression cassettes. The skilled artisan will understand that the term “expression cassette” refers to a construct of genetic material that contains coding sequences and enough regulatory information to direct proper transcription and/or translation of the coding sequences in a cell, in vivo and/or ex vivo. The expression cassette may be inserted into a vector for targeting to a desired host cell and/or into a subject or individual. Accordingly, in some embodiments, the term expression cassette may be used interchangeably with the term “expression construct.” In some embodiments, the term “expression cassette” refers to a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

In some embodiments, at least one of the expression cassettes includes a promoter operably linked to a heterologous nucleic acid sequence. Accordingly, the nucleic acid constructs as provided herein can find use, for example, as an expression vector that, when including a regulatory element (e.g., a promoter) operably linked to a heterologous nucleic acid sequence, can affect expression of the heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, at least one nonstructural protein (nsP), or a portion thereof, of the modified WEEV genome or srRNA is heterologous relative to the remainder of the modified WEEV genome or srRNA. In some embodiments, the modified WEEV genome or srRNA further includes a nucleic acid sequence encoding a heterologous nsP or a portion thereof. In some embodiments, the nucleic acid molecules of the disclosure further include one or more untranslated regions (UTRs). In some embodiments, at least one of the UTRs is a heterologous UTR.

In some embodiments, at least one of expression cassettes includes a coding sequence for a gene of interest (GOI). In some embodiments, the coding sequence for the GOI includes a coding sequence for a polypeptide construct of interest (PCI) which includes a single polypeptide (e.g., monogenic PCI). In some embodiments, the coding sequence for the PCI includes coding sequences for a plurality of polypeptides, e.g., multigenic PCI (e.g., bigenic, trigenic, or tetragenic, etc.). In some embodiments, each of the coding sequences of the plurality of polypeptides is operably linked to a separate promoter sequence. In some embodiments, the coding sequences of the plurality of polypeptides are operably linked to one another within a single open reading frame (e.g., in a polycistronic ORF). In some embodiments, the coding sequence of the polycistronic ORF is operably linked to a promoter sequence. In some embodiments, at least one of the promoter sequences is a subgenomic (sg) promoter. In some embodiments, the sg promoter is a 26S genomic promoter.

In some embodiments, the plurality of polypeptides can be linked to one another directly or indirectly (e.g., via one or more connector sequences). For example, in some embodiments, the plurality of polypeptides can be directly linked to one another, e.g., adjacently to one another. In some embodiments, at least two (e.g., 2, 3, 4, or 5) of the plurality of polypeptides are operably linked to one another by one or more connector sequences. In some embodiments, the length and amino acid composition of the connector sequences can be optimized to vary the orientation, flexibility, and/or proximity of the polypeptides relative to one another to achieve a desired activity or property of the PCI. In some embodiments, a connector sequence of the plurality of connector sequences includes one or more autoproteolytic peptide sequences. Non-limiting examples of autoproteolytic peptide sequences suitable for the methods and compositions of the disclosure include autoproteolytic cleavage sequences derived from calcium-dependent serine endoprotease (furin), porcine teschovirus-1 2A (P2A), foot-and-mouth disease virus (FMDV) 2A (F2A), Equine Rhinitis A Virus (ERAV) 2A (E2A), Thosea asigna virus 2A (T2A), cytoplasmic polyhedrosis virus 2A (BmCPV2A), and Flacherie Virus 2A (BmIFV2A). In some embodiments, at least two of the plurality of polypeptides are operably linked to each other via a P2A autoproteolytic cleavage sequence.

In some embodiments, the coding sequences of the plurality of polypeptides are operably linked to one another by one or more an internal ribosomal entry sites (IRES). Non-limiting examples of IRES suitable for the methods and compositions of the disclosure include viral IRES sequences, cellular IRES sequences, and artificial IRES sequences. Examples of suitable IRES sequences include, but are not limited to, Kaposi's sarcoma-associated herpesvirus (KSHV) IRES, hepatitis virus IRES, Pestivirus IRES, Cripavirus IRES, Rhopalosiphum padi virus IRES, fibroblast growth factor IRES, platelet-derived growth factor IRES, vascular endothelial growth factor IRES, insulin-like growth factor IRES, picornavirus IRES, encephalomyocarditis virus (EMCV) IRES, Pim-1 IRES, p53 IRES, Apaf-1 IRES, TDP2 IRES, L-myc IRES, and c-myc IRES.

In principle, there are no particular limitations with regard to suitable polypeptides and PCIs that can be expressed by the replicon constructs (e.g., srRNA constructs) of the disclosure. Exemplary types of polypeptides suitable for the compositions and methods of the disclosure include microbial proteins, viral proteins, bacterial proteins, fungal proteins, mammalian proteins, and any combinations thereof. For example, the PCI can include one or more antigen molecules and/or biotherapeutic molecules, such as cytokines, cytotoxins, chemokines, immunomodulators, pro-apoptotic factors, anti-apoptotic factors, hormones, differentiation factors, dedifferentiation factors, immune cell receptors or reporters, or combinations of any thereof.

In some embodiments, the coding sequence of the GOI is redesigned, refactored, and/or optimized for a desired property, such as increased stability, potency, and expression (e.g., translation efficiency), which in turns can maximize the impact of producing, delivering, and administering biotherapeutic. For example, in some embodiments, the coding sequence of the GOI is optimized for expression at a level higher than the expression level of a reference coding sequence, for example, 20% higher, 30% higher, 40% higher, 50% higher, 60% higher, 70% higher, 80% higher, 90% higher, or 95% higher than a reference coding sequence. In some embodiments, the reference coding sequence is a wild-type non-optimized sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure may also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence of the GOI is optimized for expression in a target host cell through the use of codons optimized for expression. The techniques for the construction of synthetic nucleic acid sequences encoding GOI using preferred codons optimal for host cell expression may be determined by computational methods analyzing the commonality of codon usage for encoding native proteins of the host cell genome and their relative abundance by techniques known in the art. Codon usage databases (e.g., http://www.kazusa.or.jp/codon) may be used for generation of codon optimized sequences in mammalian cell environments. Furthermore, a variety of software tools are available to convert sequences from one organism to the optimal codon usage for a different host organism such as the JCat Codon Optimization Tool (www.jcat.de), Integrated DNA Technologies (IDT) Codon Optimization Tool (https://www.idtdna.com/CodonOpt) or the Optimizer online codon optimization tool (http://genomes.urv.es/OPTIMIZER). Such synthetic sequences may be constructed by techniques known in the art for the construction of synthetic nucleic acid molecules and may be obtained from a variety of commercial vendors. Accordingly, in some embodiments, the coding sequence of the GOI is optimized for expression at a level higher than the expression level of a reference coding sequence, such as, for example, a coding sequence that has not been codon-optimized. In some embodiments, the codon-optimized sequence of the GOI results in an increased expression level by at least 30%, at least 40%, at least 50%, at least 60%, at least 70%, at least 80%, at least 90%, at least 95%, or at least 100% compared to a reference coding sequence that has not been codon-optimized. In some embodiments, the codon-optimized sequence of the GOI results in an increased expression level by at least 2-fold, at least 3-fold, at least 4-fold, or at least 5-fold compared to a reference coding sequence that has not been codon-optimized.

In some embodiments, the coding sequence of the GOI is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Additional information regarding principles, strategies, and methods for use in enhancing RNA stability can be found at, for example, Leppek K. et al., Combinatorial optimization of mRNA structure, stability, and translation for RNA-based therapeutics (Nature Communications. Vol 13, Article No. 1536, March 2022).

The polypeptide encoded by a GOI can generally be any polypeptide, and can be, for example a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the GOI can encode microbial proteins, viral proteins, bacterial proteins, fungal proteins, mammalian proteins, and combinations of any thereof. In some embodiments, the GOI encodes a hemagglutinin precursor (HA) of the influenza A virus H5N1. Non-limiting examples of GOI include interleukins and interacting proteins, including: G-CSF, GM-CSF, IL-1, IL-10, IL-10-like, IL-11, IL-12, IL-13, IL-14, IL-15, IL-16, IL-17, IL-18, IL-18BP, IL-1-like, IL-1RA, IL-la, IL-1B, IL-2, IL-20, IL-3, IL-4, IL-5, IL-6, IL-6-like, IL-7, IL-9, IL-21, IL-22, IL-33, IL-37, IL-38, LIF, and OSM. Additional suitable GOIs include, but are not limited to, interferons (e.g., IFN-α, IFN-β, IFN-γ), TNFs (e.g., CD154, LT-β, TNF-α, TNF-β, 4-1BBL, APRIL, CD70, CD153, CD178, GITRL, LIGHT, OX40L, TALL-1, TRAIL, TWEAK, and TRANCE), TGF-β (e.g., TGF-β1, TGF-β2, and TGF-β3), hematopoietins (e.g., Epo, Tpo, Flt-3L, SCF, M-CSF, MSP), chemokines and their receptors (e.g., XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1), immunosuppressive gene products and related transcription factors (e.g., PECAM1, FCGR3A, FOS, NFKB1, JUN, HIF1A, PD-L1, mTOR, STAT5B, and STAT4). Additional GOIs suitable for the compositions and methods of the disclosure include, but are not limited to, immunostimulatory gene products (e.g., CD27/CD70, CD40, CD40L, B7.1, BTLA, MAVS, OX40, OX40L, RIG-I, and STING), drug resistant mutants/variants of genes, such as ABCB1, ABCC1, ABCG2, AKT1, ALK, BAFF, BCR-ABL, BRAF, CCND1, cMET, EGFR, ERBB2, ERBB3, ERK2, ESR1, GRB2, KRAS, MDR1, MRP1, NTRK1, PDC4, P-gp, PI3K, PTEN, RET, ROS1, RSK1, RSK2, SHIP, and STK11. Also suitable for the compositions and methods of the disclosure includes sequence encoding viral proteins, in particular spike proteins, fiber proteins, structural proteins, and attachment proteins.

In some embodiments, the GOI can encode an antibody or antibody variant (e.g. single chain Fv, bi-specifics, camelids, Fab, and HCAb). In some embodiments, the antibody targets surface molecules associated or upregulated with cancers, or surface molecules associated with infectious disease. In some embodiments, the antibody targets surface molecules having immunostimulatory function, or having immunosuppressive function.

In some embodiments, the GOI can encode an enzyme whose deficiency or mutation is associated with diseases or health conditions, such as, for example, agalsidase beta, agalsidase alfa, imiglucerase, taliglucerase alfa, velaglucerase alfa, alglucerase, sebelipase alpha, laronidase, idursulfase, elosulfase alpha, galsulfase, alglucosidase alpha, and CTFR.

In some embodiments, the GOI can encode a polypeptide selected from antigen molecules, biotherapeutic molecules, or combinations of any thereof. In some embodiments, the GOI can encode a polypeptide selected from tumor-associated antigens, tumor-specific antigens, neoantigens, and combinations of any thereof. As will be appreciated by the skilled artisan, TAAs include a molecule, e.g., protein, present on tumor cells and on normal cells, or on many normal cells, but at much lower concentration than on tumor cells. In contrast, TSAs generally include a molecule, e.g., protein which is present on tumor cells but absent from normal cells. The tumor-associated antigen can be an antigen associated with a cancer cell, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, etc. It will also be understood that in some cases, a tumor-associated antigen may also be expressed by a non-cancerous cell. In some embodiments, the GOI can encode a polypeptide selected from estrogen receptors, intracellular signal transducer enzymes, and human epidermal growth receptors. In some embodiments, the GOI can encode a biotherapeutic polypeptide selected from immunomodulators, modulators of angiogenesis, modulators of extracellular matrix, modulators of metabolism, neurological modulators, and combinations of any thereof. In some embodiments, the GOI can encode a cytokine selected from chemokines, interferons, interleukins, lymphokines, and tumor necrosis factors. In some embodiments, the GOI can encode an interleukins selected from IL-la, IL-1B, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ and subunits of any thereof. In some embodiments, the GOI can encode a biotherapeutic polypeptide is selected from IL-12A, IL-12B, IL-1RA, and combinations of any thereof.

In some embodiments, the nucleic acid constructs of the disclosure include a nucleic acid sequence encoding a modified WEEV having at least 80%, at least 85%, at least 90%, at least 95%, at least 96%, at least 97%, at least 98%, at least 99%, or 100% sequence identity to a nucleic acid sequence of SEQ ID NO: 1. In some embodiments, the nucleic acid constructs of the disclosure include a nucleic acid sequence encoding a modified WEEV having at least 80%, at least 85%, at least 90%, at least 95%, at least 96%, at least 97%, at least 98%, at least 99%, or 100% sequence identity to a nucleic acid sequence of SEQ ID NO: 2.

In some embodiments, the nucleic acid constructs of the disclosure include a nucleic acid sequence encoding a modified WEEV having 100% sequence identity to a nucleic acid sequence of SEQ ID NO: 1, wherein one, two, three, four, five, or more nucleotides of the nucleic acid sequence may be substituted by a different nucleotide. In some embodiments, the nucleic acid constructs of the disclosure include a nucleic acid sequence encoding a modified WEEV having 100% sequence identity to a nucleic acid sequence of SEQ ID NO: 2, wherein one, two, three, four, five, or more nucleotides of the nucleic acid sequence may be substituted by a different nucleotide.

Nucleic acid sequences having a high degree of sequence identity (e.g., at least 80%, at least 85%, at least 90%, at least 95%, at least 96%, at least 97%, at least 98%, at least 99%, or 100%) to a sequence of a modified WEEV of interest can be identified and/or isolated by using the sequences identified herein (e.g., SEQ ID NO: 1 or SEQ ID NO: 2) or any others as they are known in the art, by genome sequence analysis, hybridization, and/or PCR with degenerate primers or gene-specific primers from sequences identified in the respective WEEV genome.

In some embodiments, the nucleic acid constructs of the disclosure include one or more adaptor sequences, for example, a cloning adaptor sequence. In some embodiments, the one or more adaptor sequences include the following sequence: 5′-CTGGAGACGTGGAGGAGAACCCTGGACCT-3′ (SEQ ID NO: 3). In some embodiments, the one or more adaptor sequences include the following sequence: 5′-GACCGCTACGCCCCAATGACCCGACCAGC-3′ (SEQ ID NO: 4).

In some embodiments, mutations (e.g., silent mutations) may be incorporated into the nucleic acid constructs of the disclosure to eliminate restriction enzyme cut sites, such as SapI and SpeI restriction enzyme cut sites (see, e.g., Examples 1-3). For example, a unique restriction enzyme cut site (SpeI, 5′-A′CTAG,T-3′) may be incorporated in place of the coding sequence of the native WEEV structural genes (where the 5′ A matches the location of the structural polyprotein ATG start codon, and the 3′ T matches the location of the structural polyprotein stop codon TAA). In some embodiments, a 5′ adaptor sequence (e.g., SEQ ID NO: 3) may be inserted upstream of the SpeI site, and a 3′ adaptor sequence (SEQ ID NO: 4) may be inserted downstream of the SpeI site for subsequent Gibson Assembly® procedures (Gibson et al., Nat. Methods 6, 343-345, 2009). In some embodiments, a promoter sequence (e.g., bacteriophage T7 RNA polymerase promoter) may be included upstream of the WEEV genome sequence, and downstream may contain a poly(A) sequence followed by a restriction enzyme cut site (e.g., SapI), which cuts upstream of the recognition site. Immediately downstream of this restriction enzyme cut site (SapI) may include a terminator sequence (e.g., a T7 terminator sequence) followed by a unique restriction enzyme cut site (e.g., NotI′).

In some experiments, the sequence encoding one or more of the nsPs is replaced with a heterologous nsP. In some other experiments, the sequence encoding one or more of the UTRs is replaced with a heterologous UTR.

The molecular techniques and methods by which these new nucleic acid constructs were assembled and characterized are described more fully in the Examples of the present application. In some embodiments, the nucleic acid molecules are recombinant nucleic acid molecules. As used herein, the term recombinant means any molecule (e.g. DNA, RNA, polypeptide), that is, or results, however indirect, from human manipulation. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature; 3) has been engineered using molecular cloning techniques such that it lacks one or more nucleotides with respect to the naturally occurring nucleotide sequence; and/or 4) has been manipulated using molecular cloning techniques such that it has one or more sequence changes or rearrangements with respect to the naturally occurring nucleotide sequence.

In some embodiments, the nucleic acid molecules disclosed herein are produced using recombinant DNA technology (e.g., polymerase chain reaction (PCR) amplification, cloning, etc.) or chemical synthesis. Nucleic acid molecules as disclosed herein include natural nucleic acid molecules and homologs thereof, including, but not limited to, natural allelic variants and modified nucleic acid molecules in which one or more nucleotide residues have been inserted, deleted, and/or substituted, in such a manner that such modifications provide the desired property in effecting a biological activity as described herein.

A nucleic acid molecule, including a variant of a naturally-occurring nucleic acid sequence, can be produced using a number of methods known to those skilled in the art (see, for example, Sambrook et al., In: Molecular Cloning, A Laboratory Manual, 2nd Edition, Cold Spring Harbor Press, Cold Spring Harbor, N.Y. (1989)). The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the srRNA encoded by the nucleic acid molecule and/or by hybridization with a wild-type gene or fragment thereof, or by PCR using primers having homology to a target or wild-type nucleic acid molecule or sequence.

### B. Recombinant Cells

The nucleic acid constructs of the present disclosure can be introduced into a host cell to produce a recombinant cell containing the nucleic acid molecule. Accordingly, prokaryotic or eukaryotic cells that contain a nucleic acid construct encoding a modified WEEV genome as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

Host cells can be either untransformed cells or cells that have already been transfected with at least one nucleic acid molecule. Accordingly, in some embodiments, host cells can be genetically engineered (e.g., transduced or transformed or transfected) with at least one nucleic acid molecule.

Suitable host cells for cloning or expression of the polypeptides of interest as described herein include prokaryotic or eukaryotic cells described herein. Accordingly, in some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the cell is ex vivo, e.g., has been extracted, as an individual cell or as part of an organ or tissue, from a living body or organism for a treatment or procedure, and then returned to the living body or organism. In some embodiments, the cell is in vitro, e.g., is obtained from a repository.

In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. In some embodiments, the recombinant cell is selected from the group consisting of a monkey kidney CV1 cell transformed by SV40 (e.g., COS-7), a human embryonic kidney cell (e.g., HEK 293 or HEK 293 cell), a baby hamster kidney cell (BHK), a mouse sertoli cell (e.g., TM4 cells), a monkey kidney cell (e.g., CV1), a human cervical carcinoma cell (e.g., HeLa), a canine kidney cell (e.g., MDCK), a buffalo rat liver cell (e.g., BRL 3A), a human lung cell (e.g., W138), a human liver cell (e.g., Hep G2), a mouse mammary tumor (e.g., MMT 060562), a TRI cell, a FS4 cell, a Chinese hamster ovary cell (CHO cell), an African green monkey kidney cell (e.g., Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell. In some embodiments, the recombinant cell is a cell derived from a cell described above (i.e., a derivative cell of an original cell described herein) such as, for example, a cell that is either expanded from a clone of the original cell, an engineered version of the original cell, or a reclassification of the original cell after it has undergone extensive passaging, or has been passaged through another host.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Transgenic Animals

Also provided, in another aspect, are transgenic animals including a nucleic acid construct as described herein (e.g., vector, replicon, or srRNA molecule). In some embodiments, the transgenic animal is a vertebrate animal or an invertebrate animal. In some embodiments, the transgenic animal is an insect. In some embodiments, the insect is a mosquito. In some embodiments, the transgenic animal is a mammal. In some embodiments, the transgenic mammal is a non-human mammal. Generally, transgenic animals of the present disclosure can be any non-human animal known in the art. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Additional examples of non-human animals suitable for the compositions and methods of the disclosure can include, without limitation, laboratory animals (e.g., mice, rats, hamsters, gerbils, guinea pigs, etc.), livestock (e.g., horses, cattle, pigs, sheep, goats, ducks, geese, chickens, etc.), domesticated animals and pets (e.g. cats, dogs, etc.), non-human primates (e.g., apes, chimpanzees, orangutans, monkeys, etc.), fish, amphibians (e.g., frogs, salamanders, etc.), reptiles (e.g., snakes, lizards, etc.), and other animals (e.g., foxes, weasels, rabbits, mink, beavers, ermines, otters, sable, seals, coyotes, chinchillas, deer, muskrats, possums, etc.).

In some embodiments, the transgenic animal is an insect. In some embodiments, the insect is a mosquito. In some embodiments, the transgenic animals of the present disclosure are chimeric transgenic animals. In some embodiments, the transgenic animals of the present disclosure are transgenic animals with germ cells and somatic cells containing one or more (e.g., one or more, two or more, three or more, four or more, etc.) nucleic acid constructs of the present disclosure. In some embodiments, the one or more nucleic acid constructs are stably integrated into the genome of the transgenic animals. In some embodiments, the genomes of the transgenic animals of the present disclosure can comprise any of 1, 2, 3, 4, 5, 6, 7, 8, 9, 10, 11, 12, 13, 14, 15 or more copies of the one or more nucleic acid constructs of the present disclosure.

Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298. In some embodiments, the transgenic non-human host animals of the disclosure are prepared using standard methods known in the art for introducing exogenous nucleic acid into the genome of a non-human animal. In some embodiments, the transgenic animals of the disclosure can be generated using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination. In some embodiments, the transgenic animal produces a protein of interest as described herein.

The transgenic non-human host animals of the disclosure are prepared using standard methods known in the art for introducing exogenous nucleic acid into the genome of a non-human animal. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298.

In some embodiments, the animal is a vertebrate animal or an invertebrate animal. In some embodiments, the animal is an insect. In some embodiments, the insect is a mosquito. In some embodiments, the animal is a mammalian subject. In some embodiments, the mammalian animal is a non-human animal. In some embodiments, the mammalian animal is a non-human primate. In some embodiments, the transgenic animals of the disclosure can be made using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination. In another aspect, provided herein are methods for producing a polypeptide of interest, wherein the methods include (i) rearing a transgenic animal as disclosed herein; or (ii) culturing a recombinant cell including a nucleic acid construct as disclosed herein under conditions wherein the transgenic animal or the recombinant cell produces the polypeptide encoded by the GOI. In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is an insect. In some embodiments, the insect is a mosquito. In some embodiments, the subject is a mammalian subject. In some embodiments, the mammalian subject is a human subject. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

### D. Pharmaceutical Compositions

The nucleic acid constructs, recombinant cells, recombinant polypeptides of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of a health condition such as an immune disease or a microbial infection (e.g., viral infection, micro-fungal infection, or bacterial infection). For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient.

In some embodiments, the nucleic acid constructs of the disclosure (e.g., a vectors or srRNA molecules) can be used in a naked form or formulated with a delivery vehicle. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), solid lipid nanoparticles (SLN), polyplexes, polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, IRL press (1990). Adjuvants other than liposomes and the like are also used and are known in the art. Adjuvants may protect the antigen (e.g., nucleic acid constructs, vectors, srRNA molecules) from rapid dispersal by sequestering it in a local deposit, or they may contain substances that stimulate the host to secrete factors that are chemotactic for macrophages and other components of the immune system.

The composition of the disclosure can be formulated in a format to be compatible with its intended route of administration, such as liposome, lipid-based nanoparticle (LNP), a polymer nanoparticle, a polyplex, viral replicon particle (VRP), microsphere, immune stimulating complex (ISCOM), conjugate of bioactive ligand, or a combination of any thereof. Accordingly, in some embodiments, the compositions of the disclosure that formulated in a liposome.

In some embodiments, the compositions of the disclosure that formulated in a lipid-based nanoparticle (LNP). Exemplary types of lipids suitable for the delivery systems described herein include cationic lipids, ionizable cationic lipids, anionic lipids, neutral lipids, and combinations thereof.

In some embodiments, the LNP of the disclosure can include one or more ionizable lipids. Exemplary ionizable lipids suitable for the compositions and methods of the disclosure includes those described in PCT publications WO2020252589A1 and WO2021000041A1, and Love K. T. et al., Proc Natl Acad Sci USA, Feb. 2, 2010 107 (5) 1864-1869, which are incorporated by reference herein in their entirety.

Accordingly, in some embodiments, the LNP of the disclosure includes one or more lipid compounds described in Love K. T. et al., 2010 supra, such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200.

In some embodiments, the LNP of the disclosure includes one or more cationic lipids. Suitable cationic lipids include, but are not limited to, 98N12-5, C12-200, C14-PEG2000, DLin-KC2-DMA (KC2), DLin-MC3-DMA (MC3), XTC, MD1, and 7C1.

In some embodiments, the LNP of the disclosure includes one or more neutral lipids. As described above, neural lipids, also known as “structural lipids” or “helper lipids” can also be incorporated into lipid formulations and lipid particles in some embodiments. The lipid formulations and lipid particles can include one or more structural lipids at about 10 to 40 Mol % of the composition. Suitable structural lipids support the formation of particles during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides.

Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE).

In another embodiment, the structural lipid can be any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1, which are incorporated by reference herein in their entirety.

In some embodiments, the LNP of the disclosure includes at least one lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG).

In some embodiments where the delivery systems described herein include an LNP, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

Stabilizing agents can be included in lipid formulations embodiments to ensure integrity of the mixtures. Stabilizing agents are a class of molecules which disrupt or help form the hydrophobic-hydrophilic interactions among molecules. Suitable Stabilizing agents include, but are not limited to, polysorbate 80 (also known as Tween 80, IUPAC name 2-[2-[3,4-bis(2-hydroxyethoxy)oxolan-2-yl]-2-(2-hydroxyethoxy)ethoxy]ethyl octadec-9-enoate), Myrj52 (Polyoxyethylene (40) stearate), and Brij™ S10 (Polyoxyethylene (10) stearyl ether). Polyethylene glycol conjugated lipids may also be used. The stabilizing agents may be used alone or in combinations with each other.

In some embodiments, the stabilizing agents comprises about 0.1 to 3 Mol % of the overall lipid mixture. In some embodiments, the stabilizing agents comprise about 0.5 to 2.5 Mol % of the overall lipid mixture. In some embodiments, the stabilizing agent is present at greater than 2.5 Mol %. In some embodiments the stabilizing agent is present at 5 Mol %. In some embodiments the stabilizing agent is present at 10 Mol %. In some embodiments, the stabilizing agent is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, and so forth. In other embodiments, the stabilizing agent is 2.6-10 Mol % of the lipid mixture. In other embodiments, the stabilizing agents is present at greater than 10 Mol % of the lipid mixture.

Steroids can also be included in the lipid compositions for certain applications, and lipid particles made therefrom include sterols, such as cholesterol and phytosterol.

In some embodiments, the compositions of the disclosure that formulated in a polymeric nanoparticle. Examples of polymers suitable for the compositions and methods of the disclosure can be found in Jiang X et al. (Polymeric nanoparticles for RNA delivery. Encyclopedia of Nanomaterials, 2021), which is herein incorporated by reference. Exemplary polymers suitable for the compositions and methods of the disclosure include cationic polymers, non-cationic polymers, and combinations thereof. In some embodiments, the polymeric nanoparticles of the disclosure may include a naturally-derived cationic polymer. In some embodiments, the naturally-derived cationic polymer may include chitosan, gelatin, dextran, cellulose, cyclodextrin, or a combination thereof. In some embodiments, the cationic polymer may be a synthetic cationic polymer. In some embodiments, the synthetic cationic polymer may include a polyethyleneimine (PEI), poly-L-lysine (PLL), a poly(amino acid) (PAA), a poly(amidoamine) (PAMAM), a poly(amino-co-ester) (PAE), poly(2-N,N-dimethylaminoethylmethacrylate, a poly(beta-amino ester) (PBAE), an imidazole-containing polymer, a tertiary-amine containing polymer, poly(2-(dimethylamino)ethyl methacrylate), poly-N-(2-hydroxy-propyl) methacrylamide, a polyamidoamine dendrimer, a cationic glycopolymer, or derivatives thereof.

In some embodiments, the non-cationic polymer is negatively-charged (i.e., anionic) or electronically neutral. In some embodiments, the non-cationic polymer comprises a polyethylene glycol (PEG), a polyester (e.g., polylactic acid (PLA), poly(lactic-co-glycolic acid) (PLGA), poly glycolic acid (PGA), polycaprolactone (PCL)), and polysarcosine (pSar), or derivatives thereof.

In some embodiments, the polymer may be water-soluble and/or biodegradable. In some embodiments, the polymeric nanoparticle comprises one or more of the following: poly-(?-L-glutamylglutamine) (PGGA), poly-(?-L-aspartylglutamine) (PGAA), poly-L-lactic acid (PLLA), poly-(lactic acid-co-glycolic acid) (PLGA), polyalkylcyanoacrylate (PACA), polyanhydrides, polyhydroxyacids, polypropylfumerate, polyamide, polyacetal, polyether, polyester, poly(orthoester), polycyanoacrylate, [N-(2-hydroxypropyl) methacrylamide] (HPMA) copolymer, polyvinyl alcohol, polyurethane, polyphosphazene, polyacrylate, polyurea, polyamine polyepsilon-caprolactone (PCL), and copolymers thereof.

In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant.

In some embodiments, the immunogenic compositions are formulated as a biotherapeutic, e.g., vehicle for gene delivery of different molecules with bioactivity. Non-limiting examples of biotherapeutic include cytokines, chemokines, and other soluble immunomodulators, enzymes, peptide and protein agonists, peptide and protein antagonists, hormones, receptors, antibodies and antibody-derivatives, growth factors, transcription factors, and gene silencing/editing molecules. In some embodiments, the pharmaceutical compositions are formulated as an adjuvant.

In some embodiments, the immunogenic compositions are substantially non-immunogenic or minimally immunogenic (e.g. compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intrathecal administration, intraperitoneal administration, intramuscular administration, intratracheal administration, intranodal administration, intratumoral administration, intraarticular administration, intravenous administration, subcutaneous administration, intravaginal administration, intraocular administration, rectal administration, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringeability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the pharmaceutical compositions of the disclosure are formulated for inhalation, such as an aerosol, spray, mist, liquid, or powder. Administration by inhalation may be in the form of either dry powders or aerosol formulations, which are inhaled by a subject (e.g., a patient) either through use of an inhalation device, e.g., a microspray, a pressurized metered dose inhaler, or nebulizer.

In some embodiments, the composition is formulated for one or more of intranasal administration, intrathecal administration, transdermal administration, intramuscular administration, intranodal administration, intravenous administration, intraperitoneal administration, oral administration, intravaginal, or intra-cranial administration. In some embodiments, the administered composition results in an increased production of interferon in the subject. In some embodiments, the administered composition induces production of one or more pro-inflammatory molecules in the subject. In some embodiments, the one or more pro-inflammatory molecules includes interferon gamma (IFNγ), cytokines, TNF-α, GM-CSF, and MIP1α, granzyme B, granzyme A, perforin, or a combination of any thereof.

### METHODS OF THE DISCLOSURE

Administration of any one of the therapeutic compositions described herein, e.g., nucleic acid constructs, recombinant cells, recombinant polypeptides, and/or pharmaceutical compositions, can be used in the treatment of relevant health conditions, such as proliferative disorders (e.g., cancers), infectious diseases (e.g., acute infections, chronic infections, or viral infections), rare diseases, and/or autoimmune diseases, and/or inflammatory diseases. In some embodiments, the nucleic acid constructs, recombinant cells, recombinant polypeptides, and/or pharmaceutical compositions as described herein can be useful for modulating, e.g., eliciting or suppressing a pharmacodynamic effect in a subject in need thereof. In some embodiments, the pharmacodynamic effect includes eliciting an immune response in the subject. Non-limiting examples of pharmacodynamic effect include immunogenicity effects, biomarker responses, therapeutic effects, prophylactic effects, desired effects, undesired effects, adverse effects, and effects in a disease model. Accordingly, one aspect of the disclosure relates to methods for modulating a pharmacodynamic effect in a subject in need thereof, the methods include administering to the subject a composition including one or more of the following: (a) a nucleic acid construct as described herein; (c) a recombinant cell as described herein; (c) a recombinant polypeptide as described herein; and (d) a pharmaceutical composition as described herein. In some embodiments, the pharmacodynamic effect includes one or more of the following: immunogenicity effect, a biomarker response, a therapeutic effect, a prophylactic effect, a desired effect, an undesired effect, an adverse effect, and effect in a disease model.

In some embodiments, the nucleic acid constructs, recombinant cells, recombinant polypeptides, and/or pharmaceutical compositions as described herein can be incorporated into therapeutic agents for use in methods of treating a subject who has, who is suspected of having, or who may be at high risk for developing one or more relevant health conditions or diseases. Accordingly, in another aspect, provided herein are methods for preventing or treating a health condition in a subject, the methods include prophylactically or therapeutically administering to the subject a composition including one or more of the following: (a) a self-replicating RNA construct as described herein; (b) a nucleic acid as described herein; (c) a recombinant cell as described herein; and (d) a pharmaceutical composition as described herein. In some embodiments, the administered composition elicits an immune response in the subject. In some embodiments, the administered composition induces production of one or more pro-inflammatory molecules in the subject. In some embodiments, the one or more pro-inflammatory molecules includes interferon gamma (IFNγ), cytokines, TNF-α, GM-CSF, and MIP1α, granzyme B, granzyme A, perforin, or a combination of any thereof. In some embodiments, the subject has been previously treated with one or more therapies and has developed at least a partial resistance to said one or more therapies.

Exemplary health conditions or diseases can include, without limitation, cancers, immune diseases, autoimmune diseases, inflammatory diseases, gene therapy, gene replacement, cardiovascular diseases, age-related pathologies, rare disease, acute infection, and chronic infection. In some embodiments, the subject is a patient under the care of a physician.

Examples of autoimmune diseases suitable for the methods of the disclosure include, but are not limited to, rheumatoid arthritis, osteoarthritis, Still's disease, Familiar Mediterranean Fever, systemic sclerosis, multiple sclerosis, ankylosing spondylitis, Hashimoto's tyroidism, systemic lupus erythematosus, Sjogren's syndrome, diabetic retinopathy, diabetic vasculopathy, diabetic neuralgia, insulitis, psoriasis, alopecia greata, warm and cold autoimmune hemolytic anemia (AIHA), pernicious anemia, acute inflammatory diseases, autoimmune adrenalitis, chronic inflammatory demyelinating polyneuropathy (CIDP), Lambert-Eaton syndrome, lichen sclerosis, Lyme disease, Graves disease, Behçet's disease, Ménière's disease, reactive arthritis (Reiter's syndrome), Churg-Strauss syndrome, Cogan syndrome, CREST syndrome, Pemphigus vulgaris and Pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, Sjörgensen syndrome, scleroderma, celiac disease, stiff-man syndrome, Takayasu arteritis, transient gluten intolerance, autoimmune uveitis, vitiligo, polychondritis, dermatitis herpetiformis (DH) or Duhring's disease, fibromyalgia, Goodpasture syndrome, Guillain-Barré syndrome, Hashimoto thyroiditis, autoimmune hepatitis, inflammatory bowel disease (IBD), Crohn's disease, colitis ulcerosa, myasthenia gravis, immune complex disorders, glomerulonephritis, polyarteritis nodosa, anti-phospholipid syndrome, polyglandular autoimmune syndrome, idiopatic pulmonar fibrosis, idiopathic thrombocytopenia purpura (ITP), urticaria, autoimmune infertility, juvenile rheumatoid arthritis, sarcoidosis, and autoimmune cardiomyopathy.

Non-limiting examples of infection suitable for the methods of the disclosure include infections with viruses such as human immunodeficiency virus (HIV), hepatitis B virus (HBV), hepatitis B virus (HCV), Cytomegalovirus (CMV), respiratory syncytial virus (RSV), human papillomavirus (HPV), Epstein-Barr virus (EBV), severe acute respiratory syndrome coronavirus 2 (SARS-CoV2), severe acute respiratory syndrome coronavirus (SARS-CoV), Middle East Respiratory Syndrome (MERS), influenza virus, and Ebola virus. Additional infections suitable for the methods of the disclosure include infections with intracellular parasites such as Leishmania, Rickettsia, Chlamydia, Coxiella, Plasmodium, Brucella, mycobacteria, Listeria, Toxoplasma and Trypanosoma. In some embodiments, the replicon constructs, srRNA constructs, nucleic acid constructs, recombinant cells, and/or pharmaceutical compositions, can be useful in the treatment and/or prevention of immune diseases, autoimmune diseases, or inflammatory diseases such as, for example, glomerulonephritis, inflammatory bowel disease, nephritis, peritonitis, psoriatic arthritis, osteoarthritis, Still's disease, Familiar Mediterranean Fever, systemic scleroderma and sclerosis, inflammatory bowel disease (IBD), Crohn's disease, ulcerative colitis, acute lung injury, meningitis, encephalitis, uveitis, multiple myeloma, glomerulonephritis, nephritis, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, hemolytic anemia, myasthenia gravis, lupus nephritis, lupus erythematosus, rheumatoid arthritis (RA), ankylosing spondylitis, pemphigus, Graves' disease, Hashimoto's thyroiditis, small vessel vasculitides, Omen's syndrome, chronic renal failure, autoimmune thyroid disease, acute infectious mononucleosis, HIV, herpes virus associated diseases, human virus infections, coronavirus, other enterovirus, herpes virus, influenza virus, parainfluenza virus, respiratory syncytial virus or adenovirus infection, bacteria pneumonia, wounds, sepsis, cerebral stroke/cerebral edema, ischaemia-reperfusion injury, and hepatitis C.

Non-limiting examples of inflammatory diseases suitable for the methods of the disclosure include inflammatory diseases such as asthma, inflammatory bowel disease (IBD), chronic colitis, splenomegaly, and rheumatoid arthritis.

In some embodiments, the health condition is a proliferative disorder or a microbial infection (e.g., bacterial infection, micro-fungal infection, or viral infection). In some embodiments, the subject has or is suspected of having a condition associated with proliferative disorder or a microbial infection (e.g., bacterial infection, micro-fungal infection, or viral infection).

In some embodiments, the health condition is a rare disease, e.g., a disease or condition that affects less than 200,000 people in the United States, as defined by The Orphan Drug Act (www.fda.gov/patients/rare-diseases-fda) and/or an inflammatory disorder and/or autoimmune disorder. In some embodiments, the subject has or is suspected of having a condition associated with an inflammatory disorder and/or autoimmune disorder and/or a rare disease (e.g. including but not limited to Familial Mediterranean Fever or adult onset Still's disease).

In some embodiments, the disclosed composition is formulated to be compatible with its intended route of administration. For example, the nucleic acid constructs (for example, replicon constructs, e.g., srRNA constructs), recombinant cells, recombinant polypeptides, and/or pharmaceutical compositions of the disclosure may be given orally or by inhalation, but it is more likely that they will be administered through a parenteral route. Examples of parenteral routes of administration include, for example, intramuscular, intratumoral, intraocular, intravenous, intranodal, intradermal, subcutaneous, transdermal (topical), transmucosal, intravaginal, and rectal administration. In some embodiments, the composition is administered intramuscularly. In some embodiments, the composition is administered intratumorally. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates, phosphates, tris, sucrose and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject nucleic acid constructs, recombinant cells, recombinant polypeptides, and/or pharmaceutical compositions of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions described herein, for example, nucleic acid constructs, e.g., srRNA constructs, recombinant cells, recombinant polypeptides, and/or pharmaceutical compositions, can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to nucleic acid constructs (for example, replicon constructs, e.g., srRNA constructs) and recombinant polypeptides, the therapeutically effective amount of a nucleic acid construct or recombinant polypeptide of the disclosure (e.g., an effective dosage) depends on the nucleic acid construct or recombinant polypeptide selected. For instance, single dose amounts in the range of approximately 0.001 to 0.1 mg/kg of patient body weight can be administered. In some embodiments, about 0.005, 0.01, 0.05 mg/kg may be administered. In some embodiments, single dose amounts in the range of approximately 0.03 μg to 300 μg/kg of patient body weight can be administered. In some embodiments, single dose amounts in the range of approximately 0.3 mg to 3 mg/kg of patient body weight can be administered.

As discussed supra, a therapeutically effective amount includes an amount of a therapeutic composition that is sufficient to promote a particular effect when administered to a subject, such as one who has, is suspected of having, or is at risk for a health condition, e.g., a disease or infection. In some embodiments, an effective amount includes an amount sufficient to prevent or delay the development of a symptom of the disease or infection, alter the course of a symptom of the disease or infection (for example but not limited to, slow the progression of a symptom of the disease or infection), or reverse a symptom of the disease or infection. It is understood that for any given case, an appropriate effective amount can be determined by one of ordinary skill in the art using routine experimentation.

The efficacy of a treatment including a disclosed therapeutic composition for the treatment of disease or infection can be determined by the skilled clinician. However, a treatment is considered effective treatment if at least any one or all of the signs or symptoms of disease or infection are improved or ameliorated. Efficacy can also be measured by failure of an individual to worsen as assessed by hospitalization or need for medical interventions (e.g., progression of the disease or infection is halted or at least slowed). Methods of measuring these indicators are known to those of skill in the art and/or described herein. Treatment includes any treatment of a disease or infection in a subject or an animal (some non-limiting examples include a human, or a mammal) and includes: (1) inhibiting the disease or infection, e.g., arresting, or slowing the progression of symptoms; or (2) relieving the disease or infection, e.g., causing regression of symptoms; and (3) preventing or reducing the likelihood of the development of symptoms.

In some embodiments, the nucleic acid constructs (for example, replicon constructs, e.g., srRNA constructs), recombinant cells, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments, the administered composition results in an increased production of interferon in the subject. In some embodiments of the disclosed methods, the subject is a mammal. In some embodiments, the mammal is a human subject.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringeability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the nucleic acid constructs, recombinant cells, and/or recombinant polypeptides in the required mount in an appropriate solvent with one or a combination of ingredients enumerated above, as required, followed by filtered sterilization.

When the nucleic acid constructs, recombinant cells, recombinant polypeptides, and/or pharmaceutical compositions are suitably protected, as described above, they may be orally administered, for example, with an inert diluent or an assimilable edible carrier. The nucleic acid constructs, recombinant cells, recombinant polypeptides, and/or pharmaceutical compositions and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

In some embodiments, the nucleic acid constructs and recombinant polypeptides of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

Several different ionizable cationic lipids have been developed for use in LNP. These include C12-200, MC3, LN16, and MD1 among others. For example, in one type of LNP, a GalNAc moiety is attached to the outside of the LNP and acts as a ligand for uptake in to the liver via the asialoglycoprotein receptor. Any of these cationic lipids can be used to formulate LNP for delivery of the nucleic acid constructs and recombinant polypeptides of the disclosure to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

A number of lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of cationic lipids include 98N12-5, C12-200, DLin-KC2-DMA (KC2), DLin-MC3-DMA (MC3), XTC, MD1, and 7C1. Non-limiting examples of neutral lipids include DPSC, DPPC, POPC, DOPE, and SM. Non-limiting examples of PEG-modified lipids include PEG-DMG, PEG-CerC14, and PEG-CerC20.

In some embodiments, the lipids can be combined in any number of molar ratios to produce a LNP. In addition, the polynucleotide(s) can be combined with lipid(s) in a wide range of molar ratios to produce a LNP.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having, or who may be at high risk for developing a cancer, an autoimmune disease, and/or an infection.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having, or who may be at high risk for developing a microbial infection. In some embodiments, the microbial infection is a bacterial infection. In some embodiments, the microbial infection is a fungal infection. In some embodiments, the microbial infection is a viral infection.

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein as well as written instructions for making and using the same. In particular, some embodiments of the disclosure provide kits for modulating a pharmacodynamic effect. Some embodiments of the disclosure provide kits for eliciting an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the nucleic acid constructs, recombinant cells, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs, recombinant cells, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs, recombinant cells, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers, reconstitution solutions, wash buffers, control reagents, control expression vectors, negative controls, positive controls, reagents suitable for in vitro production of the provided nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs, recombinant cells, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs, recombinant cells, and/or recombinant polypeptides of the disclosure in combination with one or more further therapeutic agents formulated together, optionally, in a pharmaceutical composition, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Construction of WEEV Vectors

This Example describes experiments performed to construct a base WEEV vector (e.g., without a heterologous gene) that are subsequently used for construction of a WEEV vector with expression of a gene of interest (e.g., hemagglutinin precursor HA pf the influenza A virus H5N1.

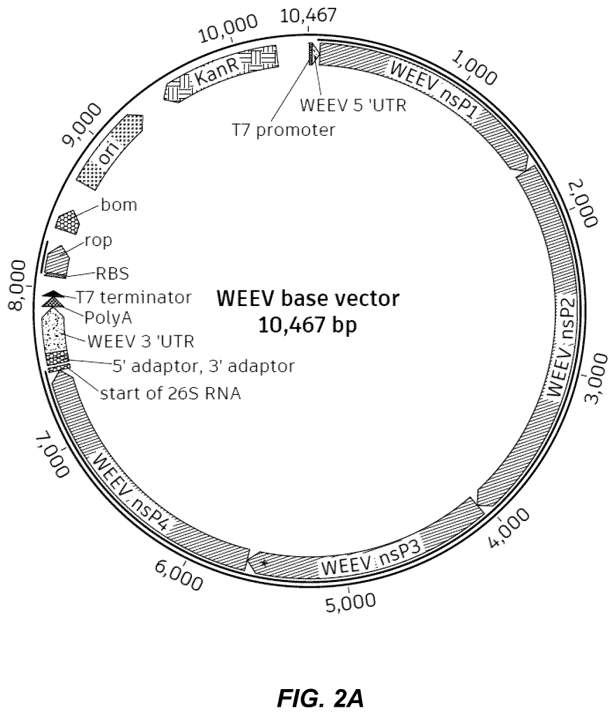

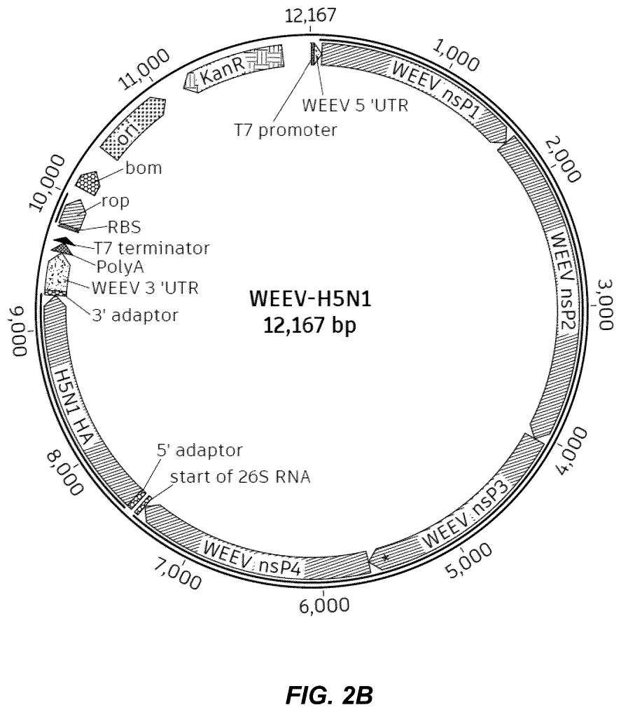

The base WEEV vector (i.e. without a heterologous gene of interest) is constructed as follows: The base WEEV vector (see, e.g., FIG. 2A) is synthesized de novo in four ˜4 kb parts from a reference sequence (Genbank GQ287641) with several modifications. Ambiguous nucleotide assignments in the reference sequence are assigned to a nucleotide which matches the synonymous codon for the encoded residue. Silent mutations are incorporated to eliminate SapI and SpeI restriction enzyme cut sites. A unique restriction enzyme cut site (SpeI, 5′-A′CTAG,T-3′) can be incorporated in place of the coding sequence of the native WEEV structural genes (where the 5′ A matches the location of the structural polyprotein ATG start codon, and the 3′ T matches the location of the structural polyprotein stop codon TAA). A 5′ adaptor sequence (5′-CTGGAGACGTGGAGGAGAACCCTGGACCT-3′; SEQ ID NO: 3) is inserted upstream of the SpeI site, and a 3′ adaptor sequence (5′-GACCGCTACGCCCCAATGACCCGACCAGC-3′; SEQ ID NO: 4) is inserted downstream of the SpeI site for subsequent Gibson Assembly® procedures (Gibson et al., Nat. Methods 6, 343-345, 2009). A bacteriophage T7 RNA polymerase promoter (5′-TAATACGACTCACTATAG-3′; SEQ ID NO: 5) is included upstream of the WEEV genome sequence, and downstream may contain a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 6) followed by a unique restriction enzyme cut site (NotI, 5′-GC′GGCC,GC-3′). The parts are combined in a five-piece Gibson Assembly® reaction: a linearized pYL backbone and the four synthesized fragments to result in the WEEV base vector.

In some experiments, the sequence encoding one or more of the nsPs is replaced with a heterologous nsP. In some other experiments, the sequence encoding one or more of the UTRs is replaced with a heterologous UTR.

Construction of a WEEV vector containing a heterologous gene are carried out as follows: the WEEV vector described in FIG. 2B is constructed by the linearization of the empty WEEV vector in FIG. 2A by SpeI digestion. The hemagglutinin (HA) gene from influenza (Genbank AY651334) is codon optimized/refactored for human expression in silico and synthesized de novo (IDT). The synthetic product is amplified using primers which added the 5′ and 3′ adaptor sequences to the end of the HA gene. The digestion product and the PCR product are combined by Gibson Assembly® procedure to result in the final vector.

### Example 2

### In Vitro Evaluation of Modified WEEV Vectors

This Example describes in vitro experiments that are performed to evaluate expression levels of the synthetic WEEV srRNA constructs described in Example 1 above, and to investigate any differential behavior thereof (e.g., replication and protein expression).

In these experiments, synthetic srRNA constructs derived from the WEEV strain Imperial are designed and subsequently evaluated.

In vitro transcription: RNA is prepared by in vitro transcription from a SapI-linearized plasmid template with bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA is then purified using phenol/chloroform extraction, or column purification (Monarch® RNA Cleanup Kit, NEB). RNA concentration is determined by absorbance at 260 nm (Nanodrop, Thermo Fisher Scientific).

Replication: RNA is transformed by electroporation into BHK-21 or Vero cells (e.g., 4D-Nucleofector™, Lonza). At 15-20 hours following transformation, the cells are fixed and permeabilized (eBioscience™ Foxp3/Transcription Factor Staining Buffer Set, Invitrogen) and subsequently stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

Protein expression: RNA is transformed by electroporation into BHK-21 or Vero cells (e.g., 4D-Nucleofector™, Lonza). At 15-20 hours following transformation, the cells are fixed and permeabilized (eBioscience™ Foxp3/Transcription Factor Staining Buffer Set, Invitrogen) and stained using an APC-conjugated anti-HA mouse monoclonal antibody (2B7, Abcam) to quantify the frequency of cells expressing the HA protein and the mean fluorescence intensity (MFI) of the HA protein in individual cells by fluorescence flow cytometry.

Additional experiments: BHK-21 or Vero cells are pre-treated with a titrated curve of recombinant interferon (IFN) prior to electroporation of RNA and impacts on replication and protein expression for vectors are measured using the assays described above.

In some experiments, BHK-21 cells are transformed with WEEV srRNA constructs. A WEEV srRNA without a target GOI is transformed by electroporation, and 20 hours following transformation, the transformed cells are fixed, permeabilized, and stained using a PE-conjugated anti-dsRNA mouse monoclonal antibody (J2, Scicons) to quantify the frequency of dsRNA+cells by fluorescence flow cytometry. In addition, a WEEV srRNA which includes the coding sequence for the target GOI is similarly transformed into BHK-21 cells, and in addition to dsRNA detection, an APC-conjugated anti-GOI monoclonal antibody was used to detect transgene expression. The positive staining of individual cells with both anti-dsRNA and anti-GOI antibodies demonstrates that the modified WEEV designs described herein are viable synthetic srRNAs and able to undergo RNA replication and express transgenes.

### Example 3

### In Vivo Evaluation of Modified WEEV Vectors

This Example describes in vivo experiments that are performed to evaluate immune responses following vaccination with the synthetic WEEV srRNA constructs described in Examples 1 and 2 above (e.g., both unformulated and LNP formulated vectors).

In these experiments, synthetic srRNA constructs derived from the WEEV strain Imperial are designed and subsequently evaluated.

Mice and injections. Female C57BL/6 or BALB/c mice are purchased from Envigo, Charles River Labs or Jackson Laboratories. On day of dosing, between 0.1-10 μg of material is injected intramuscularly split into both quadricep muscles. Vectors are administered either unformulated in saline, or LNP-formulated, or polymer-formulated. Animals are monitored for body weight and other general observations throughout the course of the study. For immunogenicity studies, animals are dosed on Day 0 and Day 21. Spleens are collected at Day 35, and serum is isolated at Days 14, and 35. For protein expression studies, animals are dosed on Day 0, and protein expression or bioluminescence is assessed on Days 1, 3, and/or 7. In some experiments where the WEEV srRNA constructs encode a reporter protein such as luciferase, in vivo imaging of luciferase activity is performed using an IVIS system at the indicated time points. In some experiments where the WEEV srRNA constructs encode secreted proteins, systemic levels are assayed by serum ELISAs.

LNP formulation. In some experiments, srRNA is formulated in lipid nanoparticles using a microfluidics mixer and analyzed for particle size, polydispersity using dynamic light scattering and encapsulation efficiency. In these experiments, a wide range of molar ratios of lipids is used in formulating LNP particles. Exemplary molar ratios of lipids used in these experiments can be 35% C12-200, 46.5% Cholesterol, 2.5% PEG-2K, and 16% DOPE.

ELISpot. To measure the magnitude of HA-specific T cell responses, IFNγ ELISpot analysis is performed using Mouse IFNγ ELISpot PLUS Kit (HRP) (MabTech) as per manufacturer's instructions. In these experiments, splenocytes are isolated and resuspended to suitable concentrations such as, for example, 5×10⁶ cells/mL in media containing peptides representing T cell epitopes for the protein of interest encoded by the WEEV srRNA constructs. Also included in these experiments are one or more positive controls such as, e.g., PMA/ionomycin, as well as DMSO which is used as a mock stimulation.

Antibodies. Antibody responses to measure total HA-specific IgG are measured using ELISA kits from Alpha Diagnostic International as per manufacturer's instructions. While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A nucleic acid construct comprising a nucleic acid sequence encoding a modified Western equine encephalitis virus (WEEV) genome or self-replicating RNA (srRNA), wherein the modified WEEV genome or srRNA is devoid of at least a portion of the nucleic acid sequence encoding one or more viral structural proteins.

2. The nucleic acid construct of claim 1, wherein the modified viral genome or srRNA is devoid of a substantial portion of the nucleic acid sequence encoding one or more viral structural proteins.

3. The nucleic acid construct of any one of claims 1-2, wherein the modified viral genome or srRNA comprises no nucleic acid sequence encoding viral structural proteins.

4. The nucleic acid construct of any one of claims 1-3, further comprising one or more expression cassettes, wherein each of the expression cassettes comprises a promoter operably linked to a heterologous nucleic acid sequence.

5. The nucleic acid construct of claim 4, wherein at least one of the expression cassettes comprises a subgenomic (sg) promoter operably linked to a heterologous nucleic acid sequence.

6. The nucleic acid construct of claim 5, wherein the sg promoter is a 26S subgenomic promoter.

7. The nucleic acid construct of any one of claims 1-6, wherein at least one nonstructural protein (nsP), or a portion thereof, of the modified WEEV genome or srRNA is heterologous relative to the remainder of the modified WEEV genome or srRNA.

8. The nucleic acid construct of any one of claims 1-7, further comprising a nucleic acid sequence encoding a heterologous nsP or a portion thereof.

9. The nucleic acid construct of any one of claims 1-8, further comprising one or more untranslated regions (UTRs).

10. The nucleic acid construct of claim 9, wherein at least one of the UTRs is a heterologous UTR.

11. The nucleic acid construct of any one of claims 4-10, wherein at least one of the expression cassettes comprises a coding sequence for a gene of interest (GOI).

12. The nucleic acid construct of claim 11, wherein the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide.

13. The nucleic acid construct of any one of claims 11-12, wherein the GOI encodes a polypeptide selected from the group consisting of an antibody, an antigen, an immune modulator, an enzyme, a signaling protein, and a cytokine.

14. The nucleic acid construct of any one of claims 11-13, wherein the coding sequence of the GOI is optimized for expression at a level higher than the expression level of a reference coding sequence.

15. The nucleic acid construct of any one of claims 11-14, wherein the coding sequence of the GOI is optimized for enhanced RNA stability.

16. The nucleic acid construct of any one of claims 1-15, wherein the nucleic acid sequence has at least 80%, at least 85%, at least 90%, at least 95%, at least 96%, at least 97%, at least 98%, at least 99%, or 100% sequence identity to the nucleic acid sequence of SEQ ID NO: 1 or SEQ ID NO: 2.

17. A recombinant cell comprising a nucleic acid construct according to any one of claims 1-16.

18. The recombinant cell of claim 17, wherein the recombinant cell is a eukaryotic cell.

19. The recombinant cell of claim 18, wherein the recombinant cell is an animal cell.

20. The recombinant cell of claim 19, wherein the animal cell is a vertebrate animal cell or an invertebrate animal cell.

21. The recombinant cell of claim 20, wherein the recombinant cell is an insect cell.

22. The recombinant cell of claim 21, wherein the recombinant cell is a mosquito cell.

23. The recombinant cell of claim 20, wherein the recombinant cell is a mammalian cell.

24. The recombinant cell of claim 20, wherein the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), a canine kidney cell (MDCK), a buffalo rat liver cell (BRL 3A), a human lung cell (W138), a human liver cell (Hep G2), a mouse mammary tumor (MMT 060562), a TRI cell, a FS4 cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

25. A cell culture comprising at least one recombinant cell according to any one of claims 17-24, and a culture medium.

26. A transgenic animal comprising a nucleic acid construct according to any one of claims 1-16.

27. The transgenic animal of claim 26, wherein the animal is a vertebrate animal or an invertebrate animal.

28. The transgenic animal of claim 26, wherein the animal is an insect.

29. The transgenic animal of claim 27, wherein the animal is a mammalian.

30. The transgenic animal of claim 29, wherein the mammalian is a non-human mammalian.

31. A method for producing a polypeptide of interest, comprising (i) rearing a transgenic animal according to any one of claims 26-30, or (ii) culturing a recombinant cell comprising a nucleic acid construct according to any one of claims 12-16 under conditions wherein the transgenic animal or the recombinant cell produces the polypeptide encoded by the GOI.

32. A method for producing a polypeptide of interest in a subject, comprising administering to the subject a nucleic acid construct according to any one of claims 12-16.

33. The method of any one of claims 29-32, wherein the subject is vertebrate animal or an invertebrate animal.

34. The method of any one of claims 29-31, wherein the subject is an insect.

35. The method of any one of claims 29-33, wherein the subject is a mammalian subject.

36. The method of claim 35, wherein the mammalian subject is a human subject.

37. A recombinant polypeptide produced by the method of any one of claims 29-36.

38. A pharmaceutical composition comprising a pharmaceutically acceptable excipient and:
a) a nucleic acid construct of any one of claims 1-16;
b) a recombinant cell of any one of claims 17-24; and/or
c) a recombinant polypeptide of claim 37.

39. The pharmaceutical composition of claim 38, comprising a nucleic acid construct of any one of claims 1-16, and a pharmaceutically acceptable excipient.

40. The pharmaceutical composition of claim 38, comprising a recombinant cell of any one of claims 17-24, and a pharmaceutically acceptable excipient.

41. The pharmaceutical composition of claim 38, comprising a recombinant polypeptide of claim 37, and a pharmaceutically acceptable excipient.

42. The pharmaceutical composition of any one of claims 38-41, wherein the composition is formulated in a liposome, a lipid-based nanoparticle (LNP), a polymer nanoparticle, a polyplex, a viral replicon particle (VRP), a microsphere, an immune stimulating complex (ISCOM), a conjugate of bioactive ligand, or a combination of any thereof.

43. The pharmaceutical composition of any one of claims 38-42, wherein the composition is an immunogenic composition.

44. The pharmaceutical composition of claim 43, wherein the immunogenic composition is formulated as a vaccine.

45. The pharmaceutical composition of any one of claims 38-42, wherein the composition is substantially non-immunogenic to a subject.

46. The pharmaceutical composition of any one of claims 38-45, wherein the pharmaceutical composition is formulated as an adjuvant.

47. The pharmaceutical composition of any one of claims 38-46, wherein the pharmaceutical composition is formulated for one or more of intranasal administration, intrathecal administration, transdermal administration, intraperitoneal administration, intramuscular administration, intratracheal administration, intranodal administration, intratumoral administration, intraarticular administration, intravenous administration, subcutaneous administration, intravaginal administration, intraocular administration, rectal administration, and oral administration.

48. A method for eliciting a pharmacodynamic effect in a subject in need thereof, the method comprises administering to the subject a composition comprising:
a) a nucleic acid construct of any one of claims 1-16;
b) a recombinant cell of any one of claims 17-24;
c) a recombinant polypeptide of claim 37; and/or
d) a pharmaceutical composition of any one of claims 38-47.

49. The method of claim 48, wherein the pharmacodynamic effect comprises one or more of the following: immunogenicity effect, a biomarker response, a therapeutic effect, a prophylactic effect, a desired effect, an undesired effect, an adverse effect, and effect in a disease model.

50. The method of claim 49, wherein the pharmacodynamic effect comprises eliciting an immune response in the subject.

51. A method for preventing and/or treating a health condition in a subject in need thereof, the method comprises prophylactically or therapeutically administering to the subject a composition comprising:
a) a nucleic acid construct of any one of claims 1-16;
b) a recombinant cell of any one of claims 17-24;
c) a recombinant polypeptide of claim 37; and/or
d) a pharmaceutical composition of any one of claims 38-47.

52. The method of claim 51, wherein the administered composition elicits an immune response in the subject.

53. The method of any one of claims 51-52, wherein the condition is a proliferative disorder or a microbial infection.

54. The method of any one of claims 51-53, wherein the subject has or is suspected of having a condition associated with proliferative disorder or a microbial infection.

55. The method of any one of claims 51-54, wherein the administered composition results in an increased production of interferon in the subject.

56. The method of any one of claims 51-55, wherein the composition is administered to the subject individually as a single therapy (monotherapy) or as a first therapy in combination with at least one additional therapies.

57. The method of claim 56, wherein the at least one additional therapies is selected from the group consisting of chemotherapy, radiotherapy, immunotherapy, hormonal therapy, toxin therapy, targeted therapy, and surgery.

58. A kit for eliciting a pharmacodynamic effect, eliciting an immune response, and/or for the prevention and/or treatment of a health condition or a microbial infection, the kit comprising:
a) a nucleic acid construct of any one of claims 1-16;
b) a recombinant cell of any one of claims 17-24;
c) a recombinant polypeptide of claim 37; and/or
d) a pharmaceutical composition of any one of claims 38-47.
and instructions for performing the method of any one of claims 48 to 57.
